(19) United States
(12) Patent Application Publication
(10) Pub. No.: US 2019/0375847 A1
(43) Pub. Date: Dec. 12, 2019

(54) COMBINATION TREATMENT FOR CANCER

(71) Applicants: GLAXOSMITHKLINE INTELLECTUAL PROPERTY DEVELOPMENT LIMITED, Brentford, Middlesex (GB); BOARD OF REGENTS, THE UNIVERSITY OF TEXAS SYSTEM, Austin, TX (US)

(72) Inventors: Maria Angelica CORTEZ, Houston, TX (US); Sharareh NIKNAM, Houston, TX (US); Jonathan E. SCHOENHALS, Houston, TX (US); James W. WELSH, Houston, TX (US); Niranjan YANAMANDRA, Collegeville, PA (US)

(21) Appl. No.: 16/485,270

(22) PCT Filed: Feb. 13, 2018
(86) PCT No.: PCT/IB2018/050882

§ 371 Date: Aug. 12, 2019

Related U.S. Application Data
(60) Provisional application No. 62/459,213, filed on Feb. 15, 2017.

(51) Int. Cl. C07K 16/28; A61P 35/00

(52) U.S. Cl. CPC C07K 16/2878; A61K 45/06; A61P 35/00; C07K 16/2818

(57) ABSTRACT

Disclosed herein is a method of treatment involving the combination of an anti-OX40 antigen binding protein (e.g., an anti-OX40 agonist antibody) and/or radiotherapy, for use in treating a cancer. The cancer may include anti-PD-1 resistance.

### CROSS REFERENCE TO RELATED APPLICATIONS

This application claims priority to U.S. Application Ser. No. 62/459,213, filed on Feb. 15, 2017. The disclosure of the prior application is considered part of (and is incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Feb. 9, 2018.

### FIELD OF THE INVENTION

The present invention relates, in part, to a method of treating a cancer in a mammal, particularly treating an anti-PD-1 resistant cancer. In particular, the present invention relates to a combination of an anti-OX40 antigen binding protein (ABP), such as an antibody (e.g., agonist antibody) to human OX40 and radiotherapy, and/or an anti-PD-1 ABP (e.g., antagonist antibody), for treating a cancer, e.g., an anti-PD-1 resistant cancer.

### BACKGROUND OF THE INVENTION

OX40 is a potent co-stimulatory receptor that can potentiate T-cell receptor signaling on the surface of T lymphocytes, leading to their activation by a specifically recognized antigen. In particular, OX40 engagement by ligands present on dendritic cells dramatically increases the proliferation, effector function and survival of T cells. Preclinical studies have shown that OX40 agonists increase anti-tumor immunity and improve tumor-free survival.

### SUMMARY OF THE INVENTION

The disclosure relates, in part, to the ability of a combination of an anti-OX40 agonist ABP and radiotherapy to treat a cancer in a subject (e.g., patient) (e.g., mammal, e.g., human), particularly in a subject that has a cancer that is anti-PD-1 resistant (i.e., a cancer with anti-PD-1 resistance).

Provided herein is a method of treating a cancer, e.g., an anti-PD-1 resistant cancer (e.g., a cancer with anti-PD-1 resistance) in a subject, the method comprising administering a combination comprising an anti-OX40 ABP, e.g., an agonist anti-OX40 ABP, and radiotherapy (e.g., therapeutically effective amounts thereof) to the subject, thereby treating the cancer. E.g., wherein the anti-OX40 ABP is an anti-OX40 ABP described herein.

Provided herein are combinations comprising an anti-OX40 ABP, e.g., an agonist anti-OX40 ABP and radiotherapy (e.g., therapeutically effective amounts thereof) for treating a cancer, e.g., an anti-PD-1 resistant cancer (e.g., a cancer with anti-PD-1 resistance). E.g., wherein the anti-OX40 ABP is an anti-OX40 ABP described herein.

Further provided is an anti-OX40 ABP, e.g., an agonist anti-OX40 ABP (e.g., a therapeutically effective amount thereof), for use in the manufacture of a medicament for the treatment of a cancer, e.g., an anti-PD-1 resistant cancer in combination (simultaneously or sequentially (e.g., in any order)) with radiotherapy (e.g., a therapeutically effective amount thereof). E.g., wherein the anti-OX40 ABP is an anti-OX40 ABP described herein.

The combination of an anti-OX40 ABP (e.g., a therapeutically effective amount thereof) and radiotherapy (e.g., a therapeutically effective amount thereof) may sensitize an anti-PD-1 resistant cancer to anti-PD1 therapy (e.g., with an anti-PD-1 ABP, e.g., antagonist anti-PD-1 ABP, e.g., antibody, e.g., wherein the anti-PD-1 ABP is an anti-PD-1 ABP described herein). E.g., wherein the anti-OX40 ABP is an anti-OX40 ABP described herein.

The combination of an anti-OX40 ABP (e.g., a therapeutically effective amount thereof) and radiotherapy (e.g., a therapeutically effective amount thereof) may cause an abscopal effect, e.g., of an anti-PD-1 resistant cancer. The cancer may be, e.g., lung cancer or melanoma.

In some aspects, provided is a combination of an anti-OX40 ABP (e.g., a therapeutically effective amount thereof) and an anti-PD-1 ABP (e.g., a therapeutically effective amount thereof), e.g., an antagonist anti-PD-1 ABP, in a method of treating a cancer, e.g., an anti-PD-1 resistant cancer in combination (simultaneously or sequentially (e.g., in any order)) with radiotherapy (e.g., a therapeutically effective amount thereof). E.g., wherein the anti-OX40 ABP is an anti-OX40 ABP described herein. E.g., wherein the anti-PD-1 ABP is an anti-PD-1 ABP described herein.

Also provided is an anti-OX40 ABP (e.g., a therapeutically effective amount thereof), e.g., further comprises an anti-PD-1 ABP (e.g., a therapeutically effective amount thereof), e.g., an antagonist anti-PD-1 ABP, for use in the manufacture of a medicament for the treatment of a cancer, e.g., an anti-PD-1 resistant cancer. E.g., wherein the anti-OX40 ABP is an anti-OX40 ABP described herein. E.g., wherein the anti-PD-1 ABP is an anti-PD-1 ABP described herein.

In some embodiments, the combination of an anti-OX40 ABP (e.g., a therapeutically effective amount thereof) and radiotherapy (e.g., a therapeutically effective amount thereof), e.g., further comprises an anti-PD-1 ABP (e.g., a therapeutically effective amount thereof), e.g., an antagonist anti-PD-1 ABP, for treating a cancer, e.g., an anti-PD-1 resistant cancer. E.g., wherein the anti-OX40 ABP is an anti-OX40 ABP described herein. E.g., wherein the anti-PD-1 ABP is an anti-PD-1 ABP described herein.

Also provided are methods of treating a cancer, e.g., an anti-PD-1 resistant cancer in a subject (e.g., human), comprising administering a combination of the invention, and uses of the combinations for therapy, preferably for therapy for a cancer, e.g., an anti-PD-1 resistant cancer.

In some aspects, the disclosure provides a method of treating a cancer in a mammal in need thereof, the method comprising: administering to the mammal an anti-OX40 antigen binding protein (e.g., a therapeutically effective amount thereof) and radiotherapy (e.g., a therapeutically effective amount thereof), thereby treating the cancer. E.g., wherein the anti-OX40 ABP is an anti-OX40 ABP described herein.

In some embodiments, the cancer is a solid tumor.

In some embodiments, the cancer is anti-PD-1 resistant.

In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a lung cancer.

In some embodiments, the cancer is a melanoma.

In some embodiments, the anti-OX40 antigen binding protein and the radiotherapy are administered at the same time.

In some embodiments, the anti-OX40 antigen binding protein is administered after the radiotherapy is administered.

In some embodiments, the anti-OX40 antigen binding protein is administered before the radiotherapy is administered.

In some embodiments, the anti-OX40 antigen binding protein is administered systemically.

In some embodiments, the anti-OX40 antigen binding protein is administered intratumorally.

In some embodiments, the mammal is human.

In some embodiments, the size of the cancer in the mammal is reduced by more than the additive amount by which the size is reduced with treatment with the anti-OX40 antigen binding protein used as a monotherapy and the radiotherapy used as a monotherapy.

In some embodiments, the anti-OX40 antigen binding protein binds to human OX40.

In some embodiments, the radiotherapy comprises external-beam radiation therapy, internal radiation therapy (brachytherapy), or systemic radiation therapy.

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

In some embodiments, the method of treatment causes an abscopal effect.

In some embodiments, the method further comprises administering to the mammal an anti-PD-1 antigen binding protein (e.g., a therapeutically effective amount thereof). E.g., wherein the anti-PD-1 ABP is an anti-PD-1 ABP described herein.

In some embodiments, the anti-PD-1 antigen binding protein binds to human PD-1.

In some embodiments, the anti-OX40 antigen binding protein and/or the anti-PD-1 antigen binding protein is a humanized monoclonal antibody.

In some embodiments, the anti-OX40 antigen binding protein and/or the anti-PD-1 antigen binding protein is a fully human monoclonal antibody.

In some embodiments, the anti-OX40 antigen binding protein and/or the anti-PD-1 antigen binding protein is an antibody with an IgG1 antibody isotype or variant thereof.

In some embodiments, the anti-OX40 antigen binding protein and/or the anti-PD-1 antigen binding protein is an antibody with an IgG4 antibody isotype or variant thereof.

In some embodiments, the anti-OX40 antigen binding protein comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the anti-OX40 antigen binding protein comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 or 17.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain variable region comprising the amino acid sequence set forth in SEQ ID NO:17 and a light chain variable region comprising the amino acid sequence set forth in SEQ ID NO:23.

In some embodiments, the anti-OX40 antigen binding protein comprises a light chain variable region comprising the amino acid sequence of SEQ ID NO:11 or 23, or an amino acid sequence with at least 90%, 91%, 92%, 93%, 94%, 95%, 96%, 97%, 98%, 99% or 100% sequence identity to the amino acid sequences of SEQ ID NO:11 or 23.

In some embodiments, the anti-OX40 antigen binding protein comprises a heavy chain variable region comprising the amino acid sequence of SEQ ID NO:5 or 17, or an amino acid sequence with at least 90%, 91%, 92%, 93%, 94%, 95%, 96%, 97%, 98%, 99% or 100% sequence identity to the amino acid sequences of SEQ ID NO:5 or 17.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the anti-PD-1 antigen binding protein is pembrolizumab (HC SEQ ID NO:50, LC SEQ ID NO:51), or an antibody comprising 90%, 91%, 92%, 93%, 94%, 95%, 96%, 97%, 98%, 99% or 100% sequence identity thereto.

In some embodiments, the anti-PD-1 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the anti-PD-1 antigen binding protein is nivolumab (HC SEQ ID NO:98, LC SEQ ID NO:99), or an antibody having 90%, 91%, 92%, 93%, 94%, 95%, 96%, 97%, 98%, 99% or 100% sequence identity thereto.

In some embodiments, the anti-PD-1 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:98 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:99.

In some embodiments, the mammal has increased survival when treated with a therapeutically effective amount of an anti-OX40 antigen binding protein in combination with radiotherapy compared with a mammal who received the anti-OX40 antigen binding protein as a monotherapy or the radiotherapy as a monotherapy.

In some embodiments, the method further comprises administering at least one anti-neoplastic agent to the mammal in need thereof.

In some aspects, the disclosure provides use of an anti-OX40 antigen binding protein (e.g., a therapeutically effective amount thereof) and systemic radiotherapy (e.g., a therapeutically effective amount thereof) in the manufacture of a medicament for the treatment of a cancer, and/or use of an anti-OX40 antigen binding protein (e.g., a therapeutically effective amount thereof) in the manufacture of a medicament for treating cancer in a mammal (e.g., human) in combination (simultaneously or sequentially) with radiotherapy (e.g., a therapeutically effective amount thereof). E.g., wherein the anti-OX40 ABP is an anti-OX40 ABP described herein.

In some embodiments, the cancer is an anti-PD-1 resistant cancer.

In some embodiments, the use causes an abscopal effect.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the medicament further comprises an anti-PD-1 antigen binding protein (e.g., a therapeutically effective amount thereof). E.g., wherein the anti-PD-1 ABP is an anti-PD-1 ABP described herein.

In some aspects, the disclosure provides an anti-OX40 antigen binding protein (e.g., a therapeutically effective amount thereof) and radiotherapy (e.g., a therapeutically effective amount thereof) for use (e.g., for simultaneous or sequential use) in treating a cancer in a mammal (e.g., human). E.g., wherein the anti-OX40 ABP is an anti-OX40 ABP described herein.

In some embodiments, the cancer is an anti-PD-1 resistant cancer.

In some embodiments, use of the combination causes an abscopal effect.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the use further comprises an anti-PD-1 antigen binding protein (e.g., a therapeutically effective amount thereof). E.g., wherein the anti-PD-1 ABP is an anti-PD-1 ABP described herein.

In some aspects, the disclosure provides a method of reducing tumor size in a mammal (e.g., human) having a cancer, the method comprising: administering an anti-OX40 antigen binding protein (e.g., a therapeutically effective amount thereof) and radiotherapy (e.g., a therapeutically effective amount thereof) to the mammal. E.g., wherein the anti-OX40 ABP is an anti-OX40 ABP described herein.

In some embodiments, the tumor comprises an anti-PD-1 resistant cancer.

In some embodiments, the method causes an abscopal effect.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the method further comprises administering to the mammal an anti-PD-1 antigen binding protein (e.g., a therapeutically effective amount thereof). E.g., wherein the anti-PD-1 ABP is an anti-PD-1 ABP described herein.

In some aspects, the disclosure provides use of an anti-OX40 antigen binding protein (e.g., therapeutically effective amount) and systemic radiotherapy (e.g., a therapeutically effective amount thereof) in the manufacture of a medicament for reducing tumor size in a mammal (e.g., human) having a cancer, and/or use of an anti-OX40 antigen binding protein (e.g., therapeutically effective amount) in the manufacture of a medicament for reducing tumor size in a mammal (e.g., human) having a cancer in combination (simultaneously or sequentially) with radiotherapy. E.g., wherein the anti-OX40 ABP is an anti-OX40 ABP described herein.

In some embodiments, the tumor comprises an anti-PD-1 resistant cancer.

In some embodiments, the use causes an abscopal effect.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the medicament further comprises an anti-PD-1 antigen binding protein (e.g., a therapeutically effective amount thereof). E.g., wherein the anti-PD-1 ABP is an anti-PD-1 ABP described herein.

In some aspects, the disclosure provides a combination of an anti-OX40 antigen binding protein (e.g., a therapeutically effective amount thereof) and radiotherapy (e.g., a therapeutically effective amount thereof) for use in reducing tumor size in a mammal (e.g., a human) having a cancer. E.g., wherein the anti-OX40 ABP is an anti-OX40 ABP described herein.

In some embodiments, the tumor comprises an anti-PD-1 resistant cancer.

In some embodiments, the combination causes an abscopal effect.

In some embodiments, the anti-OX40 antigen binding protein comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the anti-OX40 antigen binding protein comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:11 and a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:5.

In some embodiments, the anti-OX40 antigen binding protein comprises a heavy chain variable region comprising the amino acid sequence set forth in SEQ ID NO:5 and a light chain variable region comprising the amino acid sequence set forth in SEQ ID NO:11.

In some embodiments, the anti-OX40 antigen binding protein comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to amino acid sequence as set forth in SEQ ID NO:49.

In some embodiments, the combination further comprises an anti-PD-1 antigen binding protein (e.g., a therapeutically effective amount thereof). E.g., wherein the anti-PD-1 ABP is an anti-PD-1 ABP described herein.

In some aspects, the disclosure provides a kit for use in the treatment of cancer comprising:

(i) an anti-OX40 antigen binding protein;

(ii) a systemic radiotherapy; and

(iii) instructions for use in the treatment of cancer.

E.g., wherein the anti-OX40 ABP is an anti-OX40 ABP described herein.

In some embodiments, the anti-OX40 antigen binding protein and the systemic radiotherapy are each individually formulated with one or more pharmaceutically acceptable carriers.

In some aspects, the disclosure provides a kit for use in the treatment of cancer comprising:

(i) an anti-OX40 antigen binding protein; and

(iii) instructions for use in the treatment of cancer when combined with radiotherapy.

E.g., wherein the anti-OX40 ABP is an anti-OX40 ABP described herein.

In some aspects, the disclosure provides a kit for use in the treatment of cancer comprising:

(i) an anti-OX40 antigen binding protein;

(ii) an anti-PD-1 antigen binding protein;

(iii) a systemic radiotherapy; and

(iv) instructions for use in the treatment of cancer.

E.g., wherein the anti-OX40 ABP is an anti-OX40 ABP described herein. E.g., wherein the anti-PD-1 ABP is an anti-PD-1 ABP described herein.

In some embodiments, the anti-OX40 antigen binding protein, the anti-PD-1 antigen binding protein and the systemic radiotherapy are each individually formulated with one or more pharmaceutically acceptable carriers.

In some aspects, the disclosure provides a kit for use in the treatment of cancer comprising:

(i) an anti-OX40 antigen binding protein;

(ii) an anti-PD-1 antigen binding protein; and

(iii) instructions for use in the treatment of cancer when combined with radiotherapy.

E.g., wherein the anti-OX40 ABP is an anti-OX40 ABP described herein. E.g., wherein the anti-PD-1 ABP is an anti-PD-1 ABP described herein.

In some embodiments, the anti-OX40 antigen binding protein and the anti-PD-1 antigen binding protein are each individually formulated with one or more pharmaceutically acceptable carriers.

Further provided are methods for modulating the immune response of a subject in need of cancer treatment, preferably a human, comprising administering to said subject an effective amount (e.g., a therapeutically effective amount thereof) of the combinations of an anti-OX40 ABP and radiotherapy (e.g., a therapeutically effective amount thereof) (and optionally an anti-PD-1 ABP (e.g., a therapeutically effective amount thereof)), e.g., in one or more pharmaceutical compositions.

### BRIEF DESCRIPTION OF THE DRAWINGS

FIGS. 1-12 show sequences of anti-OX40 ABPs. FIG. 1 includes a disclosure of residues 1-30, 36-49, 67-98, and 121-131 of SEQ ID NO:70. X61012 is disclosed as SEQ ID NO: 70. FIG. 2 includes a disclosure of residues 1-23, 35-49, 57-88, and 102-111 of SEQ ID NO:71. AJ388641 is disclosed as SEQ ID NO:71. FIG. 3 includes a disclosure of the amino acid sequence as SEQ ID NO:72. FIG. 4 includes a disclosure of the amino acid sequence as SEQ ID NO:73. FIG. 5 includes a disclosure of residues 17-46, 52-65, 83-114, and 126-136 of SEQ ID NO:74. Z14189 is disclosed as SEQ ID NO:74. FIG. 6 includes a disclosure of residues 21-43, 55-69, 77-108, and 118-127 of SEQ ID NO:75. M29469 is disclosed as SEQ ID NO:75. FIG. 7 includes a disclosure of the amino acid sequence as SEQ ID NO:76. FIG. 8 includes a disclosure of the amino acid sequence as SEQ ID NO:77.

FIG. 13 shows the experimental design overview.

FIGS. 14A and B show the effect of intratumoral OX86 and radiation on tumor volume in an anti-PD-1-resistant tumor model. Female 129 Sv/ev mice were inoculated with anti-PD-1 resistant cells subcutaneously (s.c.) on the right flank on day 0 and left flank on day 4. Animals were randomized and treatment started on day 9 after tumor inoculation. Mice received anti-OX40 antibody (200 μg/mouse) intratumoral injection or radiation treatments (12Gy*3) to the primary tumor. N=10 per group. FIG. 14A: primary tumor; FIG. 14B: secondary tumor.

FIG. 15 shows the combination of radiation plus OX86 decreases lung metastasis in an anti-PD-1 resistant model.

FIG. 16 shows the effect of radiation plus OX86 on survival in an anti-PD-1 resistant model.

FIGS. 17A-D show the percentage of CD4 (FIGS. 17A and C) and CD8 (FIGS. 17B and D) T Cells in primary and secondary tumors at day 32, 48 hrs post final dose of OX86.

FIGS. 18A and B show the OX40L expression on macrophages (FIG. 18A) and neutrophils (FIG. 18B) in primary tumors at day 32, 48 hrs post the final dose of OX86 or isotype.

FIGS. 19A-C show the expression of OX40 on CD4 (FIG. 19A) and CD8 (FIG. 19B) T cells and percentages of dendritic cells in spleens (FIG. 19C) 48 h after XRT 12Gy*3.

FIGS. 20A-C show the expression of OX40 on CD4 T cells spleens (FIG. 20A) and tumors (FIG. 20B) and percentages of dendritic cells in spleens (FIG. 20C) 7 days after XRT 12Gy*3.

### DETAILED DESCRIPTION OF THE INVENTION

The combination of an anti-OX40 agonist ABP and radiotherapy can be effective in treating a cancer, particularly an anti-PD-1 resistant cancer. The combination can further include an anti-PD-1 antagonist ABP. The combination of an anti-OX40 agonist ABP and radiotherapy and an anti-PD-1 antagonist ABP can be effective in treating a cancer, particularly an anti-PD-1 resistant cancer. For example, the combination of an OX40 agonist and radiotherapy may sensitize an anti-PD-1 resistant cancer to anti-PD-1 therapy.

Compositions and Combinations

An emerging immunotherapeutic strategy is to target T cell co-stimulatory molecules, e.g., OX40. OX40 (e.g., human OX40 (hOX40) or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells. Not wishing to be bound by theory, the anti-OX40 ABPs (agonist anti-OX40 ABPs) of a combination of the invention, or a method or use thereof, modulate OX40 and promote growth and/or differentiation of T cells and increase long-term memory T-cell populations, e.g., in overlapping mechanisms as those of OX40L, by “engaging” OX40. The anti-OX40 ABPs of the invention are agonist antibodies. Thus, in one embodiment of the ABPs of a combination of the invention, or a method or use thereof, bind and engage OX40. In another embodiment, the anti-OX40 ABPs of a combination of the invention, or a method or use thereof, modulate OX40. In a further embodiment, the ABPs of a combination of the invention, or a method or use thereof, modulate OX40 by mimicking OX40L. In another embodiment, the anti-OX40 ABPs of a combination of the invention, or a method or use thereof, modulate OX40 and cause proliferation of T cells. In a further embodiment, the anti-OX40 ABPs of a combination of the invention, or a method or use thereof, modulate OX40 and improve, augment, enhance, or increase proliferation of CD4 T cells. In another embodiment, the anti-OX40 ABPs of a combination of the invention, or a method or use thereof, improve, augment, enhance, or increase proliferation of CD8 T cells. In a further embodiment, the anti-OX40 ABPs of a combination of the invention, or a method or use thereof, improve, augment, enhance, or increase proliferation of both CD4 and CD8 T cells. In another embodiment, the anti-OX40 ABPs of a combination of the invention, or a method or use thereof, enhance T cell function, e.g., of CD4 or CD8 T cells, or both CD4 and CD8 T cells. In a further embodiment, the anti-OX40 ABPs of a combination of the invention, or a method or use thereof, enhance effector T cell function. In another embodiment, the anti-OX40 ABPs of a combination of the invention, or a method or use thereof, improve, augment, enhance, or increase long-term survival of CD8 T cells. In further embodiments, any of the preceding effects occur in a tumor microenvironment.

Not being bound by theory, of equal importance is the blockade of a potentially robust immunosuppressive response at the tumor site by mediators produced both by T regulatory cells (Tregs) as well as the tumor itself (e.g., Transforming Growth Factor (TGF-B) and interleukin-10 (IL-10)). Not wishing to be bound by theory, a key immune pathogenesis of cancer can be the involvement of Tregs that are found in tumor beds and sites of inflammation. In general, Treg cells occur naturally in circulation and help the immune system to return to a quiet, although vigilant state, after encountering and eliminating external pathogens. They help to maintain tolerance to self antigens and are naturally suppressive in function. They are phenotypically characterized as CD4+, CD25+, FOXP3+ cells. Not wishing to be bound by theory, but in order to break tolerance to effectively treat certain cancers, one mode of therapy is to eliminate Tregs preferentially at tumor sites. Targeting and eliminating Tregs leading to an antitumor response has been more successful in tumors that are immunogenic compared to those that are poorly immunogenic. Many tumors secrete cytokines, e.g., TGF-B that may hamper the immune response by causing precursor CD4+25+ cells to acquire the FOXP3+ phenotype and function as Tregs.

“Modulate” as used herein, for example with regard to a receptor or other target, means to change any natural or existing function of the receptor, for example it means affecting binding of natural or artificial ligands to the receptor or target; it includes initiating any partial or full conformational changes or signaling through the receptor or target, and also includes preventing partial or full binding of the receptor or target with its natural or artificial ligands. Also included in the case of membrane bound receptors or targets are any changes in the way the receptor or target interacts with other proteins or molecules in the membrane or change in any localization (or co-localization with other molecules) within membrane compartments as compared to its natural or unchanged state. Modulators are therefore compounds or ligands or molecules that modulate a target or receptor. Modulate includes agonizing, e.g., signaling, as well as antagonizing, or blocking signaling or interactions with a ligand or compound or molecule that happen in the unchanged or unmodulated state. Thus, modulators may be agonists or antagonists. Further, one of skill in the art will recognize that not all modulators will have absolute selectivity for one target or receptor, but are still considered a modulator for that target or receptor; for example, a modulator may also engage multiple targets.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor and/or (4) enhances, increases, promotes or induces the expression of the receptor. Agonist activity can be measured in vitro by various assays known in the art such as, but not limited to, measurement of cell signalling, cell proliferation, immune cell activation markers, and cytokine production. Agonist activity can also be measured in vivo by various assays that measure surrogate end points such as, but not limited to, the measurement of T cell proliferation or cytokine production.

As used herein the term “antagonist” refers to an antigen binding protein including but not limited to an antibody, which upon contact (e.g., with a co-signalling receptor) causes one or more of the following (1) attenuates, blocks or inactivates the receptor and/or blocks activation of a receptor by its natural ligand, (2) reduces, decreases or shortens the activity, function or presence of the receptor and/or (3) reduces, decreases, or abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to, the measurement of T cell proliferation or cytokine production.

Thus, in one embodiment, an agonist anti-OX40 ABP inhibits the suppressive effect of Treg cells on other T cells, e.g., within the tumor environment.

Accumulating evidence suggests that the ratio of Tregs to T effector cells in the tumor correlates with anti-tumor response. Therefore, in one embodiment, the OX40 ABPs (anti-OX40 ABPs) of a combination of the invention, or a method or use thereof, modulate OX40 to augment T effector number and function and inhibit Treg function.

Enhancing, augmenting, improving, increasing, and otherwise changing the anti-tumor effect of OX40 is an object of a combination of the invention, or a method or use thereof. Described herein are combinations of an anti-OX40 ABP, or a method or use thereof, and another therapy for cancer, e.g., radiotherapy, and/or another compound, such as a PD-1 modulator (e.g., an anti-PD-1 ABP) described herein.

Thus, as used herein the term “combination of the invention” refers to a combination comprising an anti-OX40 ABP, suitably an agonist anti-OX40 ABP, and another treatment described herein, suitably radiotherapy and/or an anti-PD-1 ABP (suitably an antagonist anti-PD-1 ABP), each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation. Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths. Tumors may be hematopoietic tumors, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The invention further provides pharmaceutical compositions, which include one or more of the components herein, and one or more pharmaceutically acceptable carriers, diluents, or excipients. The combination of the invention may comprise two pharmaceutical compositions, one comprising an anti-OX40 ABP of the invention, suitably an agonist anti-OX40 ABP, and the other comprising an anti-PD-1 ABP, suitably an antagonist anti-PD-1 ABP, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered in any order, and in different routes; the components and pharmaceutical compositions comprising the same may be administered simultaneously or sequentially.

In accordance with another aspect of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g., intravenously, and one or more other components of a combination of the invention are administered intratumorally. In another embodiment, all of the components of a combination of the invention are administered systemically, e.g., intravenously. In an alternative embodiment, all of the components of the combination of the invention are administered intratumorally. In any of the embodiments, e.g., in this paragraph, the components of the invention are administered as one or more pharmaceutical compositions.

Antigen Binding Proteins

“Antigen Binding Protein (ABP)” means a protein that binds an antigen, including antibodies or engineered molecules that function in similar ways to antibodies. Such alternative antibody formats include triabody, tetrabody, miniantibody, and a minibody. Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP of a combination of the invention, or a method or use thereof, may comprise the variable heavy chain (VH) and variable light chain (VL) regions formatted into a full length antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533 which comprises an antigen binding region and a non-immunoglobulin region.

Herein, the antigen that the anti-OX40 antigen binding protein (ABP) binds is OX40, such as human OX40. The following terms are used herein interchangeably to mean an antigen binding protein that binds to OX40: an OX40 binding protein, an OX40 ABP, an anti-OX40 antigen binding protein, an anti-OX40 ABP, an OX40 antigen binding protein, an antigen binding protein to OX40, an ABP to OX40.

Thus, herein an anti-OX40 ABP of a combination, or a method or use thereof, of the invention or protein is one that binds OX40, and in preferred embodiments does one or more of the following: modulate signaling through OX40, modulates the function of OX40, agonize OX40 signalling, stimulate OX40 function, or co-stimulate OX40 signaling. One of skill in the art would readily recognize a variety of well known assays to establish such functions.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e. produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of a combination of the invention, or a method or use thereof, which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the anti-OX40 ABPs of a combination herein, or method or use therof, of the invention bind an epitope of OX40; likewise an anti-PD-1 ABP of a combination herein, or a method or use thereof, of the invention binds an epitope of PD-1. The epitope of an ABP is the region of its antigen to which the ABP binds. Two ABPs bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Also the same epitope may include “overlapping epitopes” e.g., if some amino acid mutations that reduce or eliminate binding of one antibody reduce or eliminate binding of the other.

The strength of binding may be important in dosing and administration of an ABP of the combination, or method or use thereof, of the invention. Affinity is the strength of binding of one molecule, e.g., an antibody of a combination of the invention, or a method or use thereof, to another, e.g., its target antigen, at a single binding site. The binding affinity of an antibody to its target may be determined by equilibrium methods (e.g., enzyme-linked immunoabsorbent assay (ELISA) or radioimmunoassay (RIA)), or kinetics (e.g., BIACORE analysis). For example, the BIACORE methods known in the art may be used to measure binding affinity. In one embodiment, the ABP of the invention binds its target (e.g., OX40 or PD-1) with high affinity. For example, when measured by BIACORE, the antibody binds to OX40, preferably human OX40, with a KD of 1-1000 nM or 500 nM or less or a KD of 200 nM or less or a KD of 100 nM or less or a KD of 50 nM or less or a KD of 500 pM or less or a KD of 400 pM or less, or 300 pM or less. In a further aspect, the antibody binds to OX40, preferably human OX40, when measured by Biacore with a KD of between about 50 nM and about 200 nM or between about 50 nM and about 150 nM. In one aspect of the present invention the antibody binds OX40, preferably human OX40, with a KD of less than 100 nM. For example, when measured by BIACORE, the antibody binds to PD-1, preferably human PD-1, with a KD of 1-1000 nM or 500 nM or less or a KD of 200 nM or less or a KD of 100 nM or less or a KD of 50 nM or less or a KD of 500 pM or less or a KD of 400 pM or less, or 300 pM or less. In a further aspect, the antibody binds to PD-1, preferably human PD-1, when measured by BIACORE with a KD of between about 50 nM and about 200 nM or between about 50 nM and about 150 nM. In one aspect of the present invention the antibody binds PD-1, preferably human PD-1, with a KD of less than 100 nM. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

Avidity is the sum total of the strength of binding of two molecules to one another at multiple sites, e.g., taking into account the valency of the interaction.

The dissociation rate constant (kd) or “off-rate” describes the stability of the complex of the ABP on one hand and target (e.g., OX40 or PD-1, preferably human OX40 or human PD-1) on the other hand, i.e., the fraction of complexes that decay per second. For example, a kd of 0.01 s⁻¹ equates to 1% of the complexes decaying per second. In an embodiment, the dissociation rate constant (kd) is 1×10⁻³ s⁻¹ or less, 1×10⁻⁴ s⁻¹ or less, 1×10⁻⁵s⁻¹ or less, or 1×10⁻⁶ s⁻¹ or less. The kd may be between 1×10⁻⁵ s⁻¹ and 1×10⁻⁴ s⁻¹, or between 1×10⁻⁴ s⁻¹ and 1×10⁻³ s⁻¹.

Competition between an anti-OX40 ABP of a combination of the invention, or a method or use thereof, and a reference antibody, e.g., for binding OX40, an epitope of OX40, or a fragment of the OX40, may be determined by competition ELISA, FMAT or BIACORE. Competition between an anti-PD-1 ABP of a combination of the invention, or a method or use thereof, and a reference antibody, e.g., for binding PD-1, an epitope of PD-1, or a fragment of the PD-1, may be determined by competition ELISA, FMAT or BIACORE. In one aspect, the competition assay is carried out by BIACORE. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding OX40 or PD-1 as defined herein.

Functional fragments of the ABPs of a combination of the invention, or a method or use thereof, are contemplated herein.

Thus, “binding fragments” and “functional fragments” may be a Fab and F(ab′)2 fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. Biochemistry 12:1130-1135 (1973); Sharon, J. et al. Biochemistry 15:1591-1594 (1976)). These various fragments are produced using conventional techniques such as protease cleavage or chemical cleavage (see, e.g., Rousseaux et al., Meth. Enzymol., 121:663-69 (1986)).

“Functional fragments” as used herein means a portion or fragment of the ABPs of a combination of the invention, or a method or use thereof, that include the antigen-binding site and are capable of binding the same target as the parent ABP, e.g., but not limited to binding the same epitope, and that also retain one or more modulating or other functions described herein or known in the art.

As the ABPs of the present invention may comprise heavy chain variable regions and light chain variable regions of a combination of the invention, or a method or use thereof, which may be formatted into the structure of a natural antibody, a functional fragment is one that retains binding or one or more functions of the full length ABP as described herein. A binding fragment of an ABP of a combination of the invention, or a method or use thereof, may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term “CDR” as used herein, refers to the complementarity determining region amino acid sequences of an antigen binding protein. These are the hypervariable regions of immunoglobulin heavy and light chains. There are three heavy chain and three light chain CDRs (or CDR regions) in the variable portions of an immunoglobulin.

It will be apparent to those skilled in the art that there are various numbering conventions for CDR sequences; Chothia (Chothia et al. Nature 342:877-883 (1989)), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1” (or “LC CDR1”), “CDRL2” (or “LC CDR2”), “CDRL3” (or “LC CDR3”), “CDRH1” (or “HC CDR1”), “CDRH2” (or “HC CDR2”), “CDRH3” (or “HC CDR3”) refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In particular embodiments, the numbering convention is the Kabat convention.

The term “CDR variant” as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. It will be appreciated that each CDR that can be modified may be modified alone or in combination with another CDR. In one aspect, the modification is a substitution, particularly a conservative substitution, for example as shown in Table A.

TABLE A

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

For nucleotide and amino acid sequences, the term “identical” or “identity” indicates the degree of identity between two nucleic acid or two amino acid sequences when optimally aligned and compared with appropriate insertions or deletions.

The percent sequence identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In any embodiment of a combination of the invention, or a method or use thereof, herein, the ABP may have any one or all CDRs, VH, VL, heavy chain (HC), light chain (LC), with 99, 98, 97, 96, 95, 94, 93, 92, 91, or 90, or 85, or 80, or 75, or 70 percent identity to the sequence shown or referenced, e.g., as defined by a SEQ ID NO disclosed herein.

With respect to an antibody, the percent identity can be over the entire VL or LC sequence, or the percent identity can be confined to the non-CDR regions (e.g., framework regions) while the sequences that correspond to CDRs have 100% identity to the disclosed CDRs within the VL or LC.

With respect to an antibody, the percent identity can be over the entire VH or HC sequence, or the percent identity can be confined to the non-CDR regions (e.g., framework regions) while the sequences that correspond to CDRs have 100% identity to the disclosed CDRs within the VH or HC.

Antigen Binding Proteins that Bind OX40

ABPs that bind human OX40 (also referred to as OX-40 or OX40 receptor or OX40R) are provided herein (i.e., an anti-OX40 ABP and an anti-human OX40 receptor (hOX-40R) ABP, sometimes referred to herein as an “anti-OX40 ABP”, such as an“anti-OX40 antibody”). These ABPs, such as antibodies, are useful in the treatment or prevention of acute or chronic diseases or conditions whose pathology involves OX40 signalling. In one aspect, an antigen binding protein, or isolated human antibody or functional fragment of such protein or antibody, that binds to human OX40R and is effective as a cancer treatment or treatment against disease is described, for example in combination with radiotherapy and/or with another compound such as an anti-PD-1 ABP, suitably an antagonist anti-PD-1 ABP. Any of the antigen binding proteins or antibodies disclosed herein may be used as a medicament. Any one or more of the antigen binding proteins or antibodies may be used in the methods or compositions to treat cancer, e.g., those disclosed herein. The anti-OX40 ABPs are agonist antibodies, e.g., agonists of OX40 (i.e., of OX40 receptor).

The isolated ABPs, such as antibodies, as described herein bind to OX40, and may bind to OX40 encoded from the following genes: NCBI Accession Number NP_003317, Genpept Accession Number P23510, or genes having 90 percent homology or 90 percent identity thereto. The isolated antibody provided herein may further bind to OX40 (OX40 receptor) having one of the following GenBank Accession Numbers: AAB39944, CAE11757, or AAI05071.

Antigen binding proteins such as antibodies that bind and/or modulate OX40 (OX-40 receptor) are known in the art. Exemplary anti-OX40 ABPs of a combination of the invention, or a method or use thereof, are disclosed, for example in International Publication No. WO2013/028231 (PCT/US2012/024570), international filing date 9 Feb. 2012, and WO2012/027328 (PCT/US2011/048752), international filing date 23 Aug. 2011, each of which is incorporated by reference in its entirety herein (To the extent any definitions conflict, this instant application controls).

In one embodiment, the OX40 antigen binding protein is ANTIBODY 106-222 (HC of SEQ ID NO: 48 and LC of SEQ ID NO:49). In another embodiment, the antigen binding protein comprises the CDRs (SEQ ID NOS:1-3 and 7-9) of ANTIBODY 106-222, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH (SEQ ID NO:5), a VL (SEQ ID NO:11), or both of ANTIBODY 106-222 (i.e. humanized 106-222), or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is MEDI6469; MEDI6383; MEDI0562; MOXR0916 (RG7888); PF-04518600; BMS986178; or INCAGN01949. In another embodiment, the antigen binding protein comprises the CDRs of MEDI6469; MEDI6383; MEDI0562; MOXR0916 (RG7888); PF-04518600; BMS986178; or INCAGN01949, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of MEDI6469; MEDI6383; MEDI0562; MOXR0916 (RG7888); PF-04518600; BMS986178; or INCAGN01949, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is MEDI6469. In another embodiment, the antigen binding protein comprises the CDRs of MEDI6469, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of MEDI6469, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is MEDI6383. In another embodiment, the antigen binding protein comprises the CDRs of MEDI6383, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of MEDI6383, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is MEDI0562. In another embodiment, the antigen binding protein comprises the CDRs of MEDI0562, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of MEDI0562, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is MOXR0916 (RG7888). In another embodiment, the antigen binding protein comprises the CDRs of MOXR0916 (RG7888), or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of MOXR0916 (RG7888), or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is PF-04518600. In another embodiment, the antigen binding protein comprises the CDRs of PF-04518600, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of PF-04518600, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is BMS986178. In another embodiment, the antigen binding protein comprises the CDRs of BMS986178, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of BMS986178, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is INCAGN01949. In another embodiment, the antigen binding protein comprises the CDRs of INCAGN01949, or CDRs with at least 90% (e.g., 90%, 91%, 92%, 93%, 94%, 95%, 96%, 97%, 98%, 99%, or 100%) sequence identity to the CDR sequences thereof. In a further embodiment the antigen binding protein comprises a VH, a VL, or both of INCAGN01949, or a VH or a VL with at least 90% (e.g., 90%, 91%, 92%, 93%, 94%, 95%, 96%, 97%, 98%, 99%, or 100%) sequence identity to the VH or VL sequences thereof.

In one embodiment, the OX40 antigen binding protein is one disclosed in WO2015/153513. In another embodiment, the antigen binding protein comprises the CDRs of an antibody disclosed in WO2015/153513, or CDRs with at least 90% (e.g., 90%, 91%, 92%, 93%, 94%, 95%, 96%, 97%, 98%, 99%, or 100%) sequence identity to the disclosed CDR sequences. In a further embodiment the antigen binding protein comprises a VH, a VL, or both of an antibody disclosed in WO2015/153513, or a VH or a VL with at least 90% (e.g., 90%, 91%, 92%, 93%, 94%, 95%, 96%, 97%, 98%, 99%, or 100%) sequence identity to the disclosed VH or VL sequences.

In one embodiment, the OX40 antigen binding protein is one disclosed in WO2013/038191. In another embodiment, the antigen binding protein comprises the CDRs of an antibody disclosed in WO2013/038191, or CDRs with at least 90% (e.g., 90%, 91%, 92%, 93%, 94%, 95%, 96%, 97%, 98%, 99%, or 100%) sequence identity to the disclosed CDR sequences. In a further embodiment the antigen binding protein comprises a VH, a VL, or both of an antibody disclosed in WO2013/038191, or a VH or a VL with at least 90% (e.g., 90%, 91%, 92%, 93%, 94%, 95%, 96%, 97%, 98%, 99%, or 100%) sequence identity to the disclosed VH or VL sequences.

In one embodiment, the OX40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with at least 90% (e.g., 90%, 91%, 92%, 93%, 94%, 95%, 96%, 97%, 98%, 99%, or 100%) sequence identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with at least 90% (e.g., 90%, 91%, 92%, 93%, 94%, 95%, 96%, 97%, 98%, 99%, or 100%) sequence identity to the disclosed VH or VL sequences.

In another embodiment, the OX40 antigen binding protein is one disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with at least 90% (e.g., 90%, 91%, 92%, 93%, 94%, 95%, 96%, 97%, 98%, 99%, or 100%) sequence identity to the disclosed CDR sequences. In a further embodiment the antigen binding protein comprises a VH, a VL, or both of an antibody disclosed in WO2013/028231 (PCT/US2012/024570), international filing date 9 Feb. 2012, or a VH or a VL with at least 90% (e.g., 90%, 91%, 92%, 93%, 94%, 95%, 96%, 97%, 98%, 99%, or 100%) sequence identity to the disclosed VH or VL sequences.

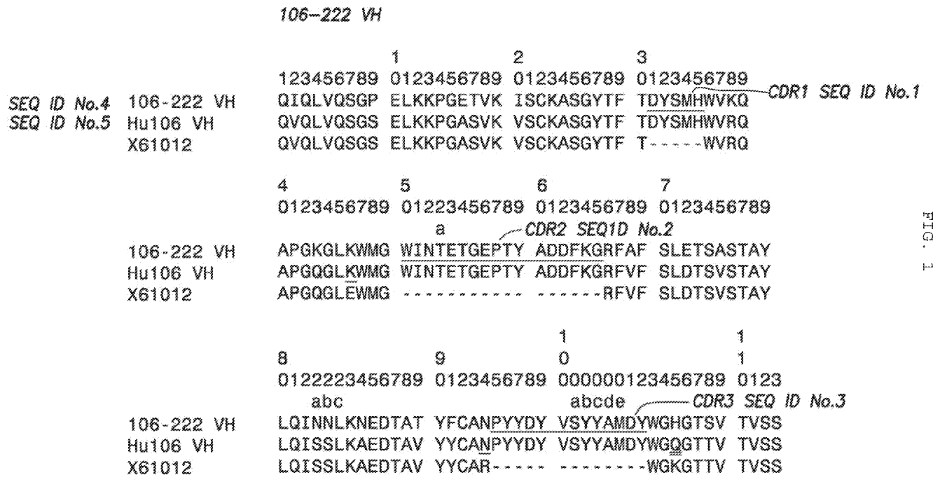

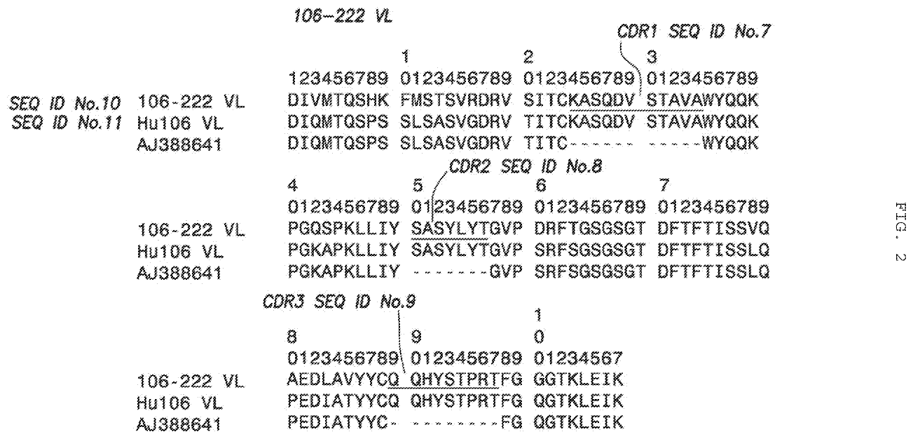

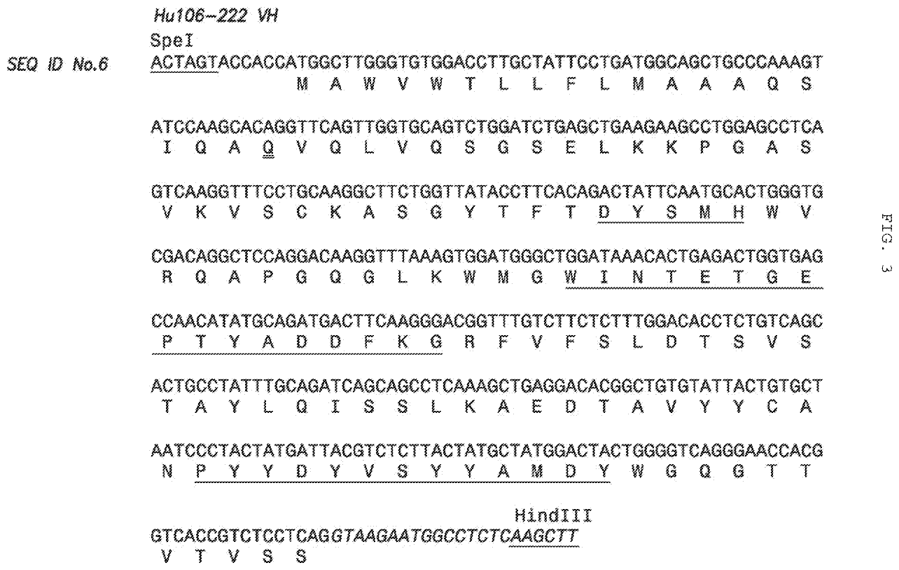

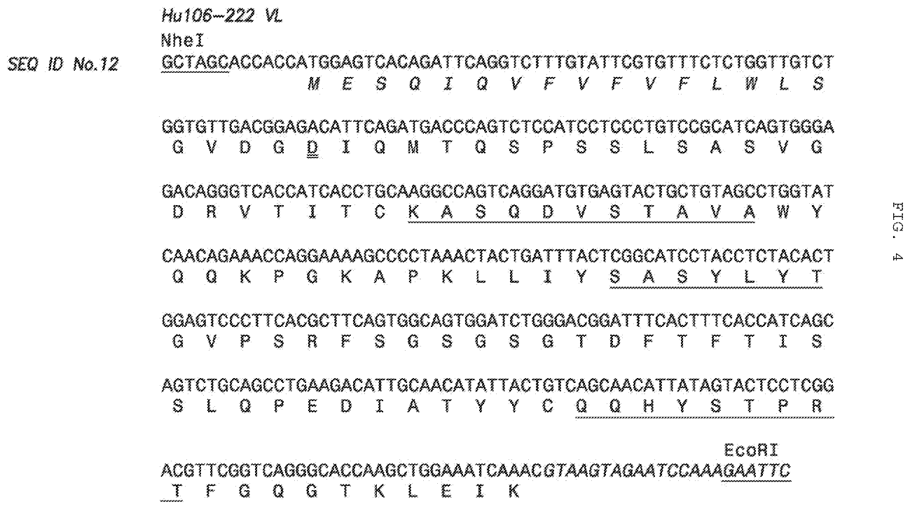

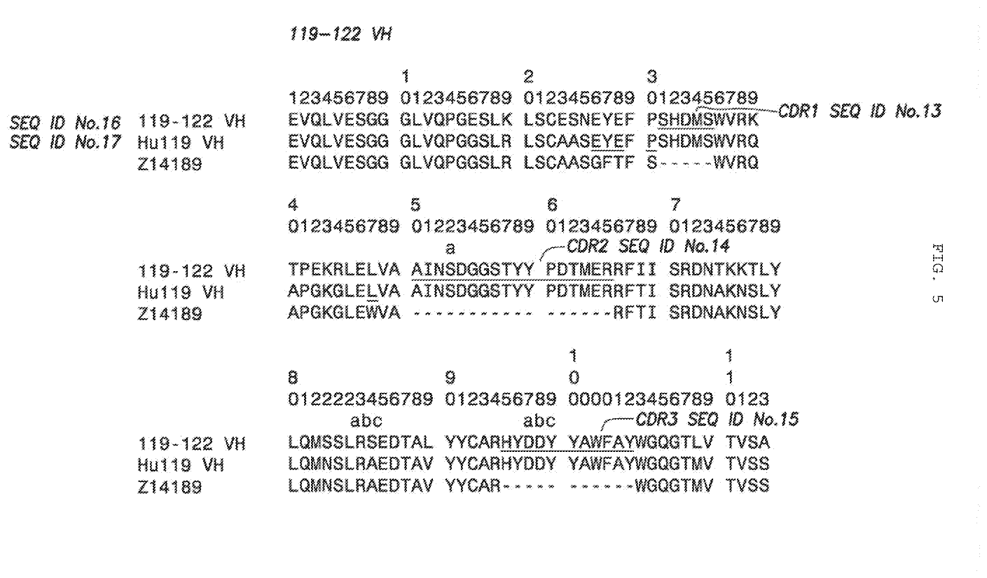

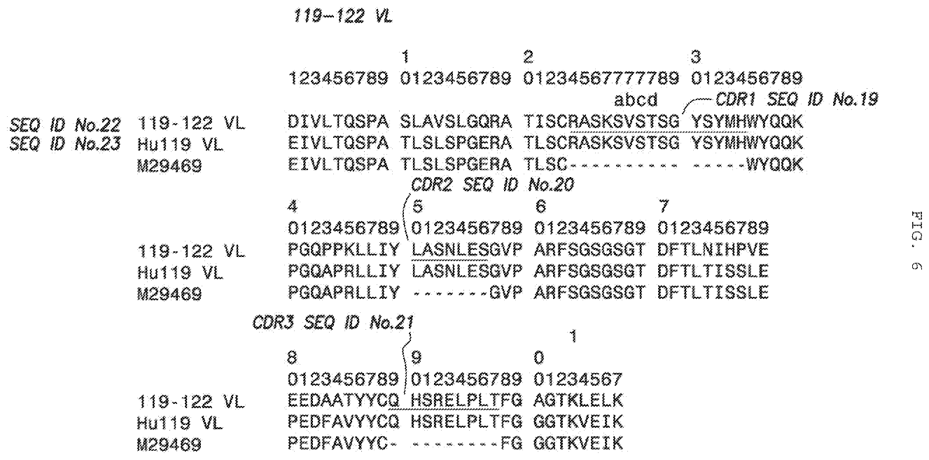

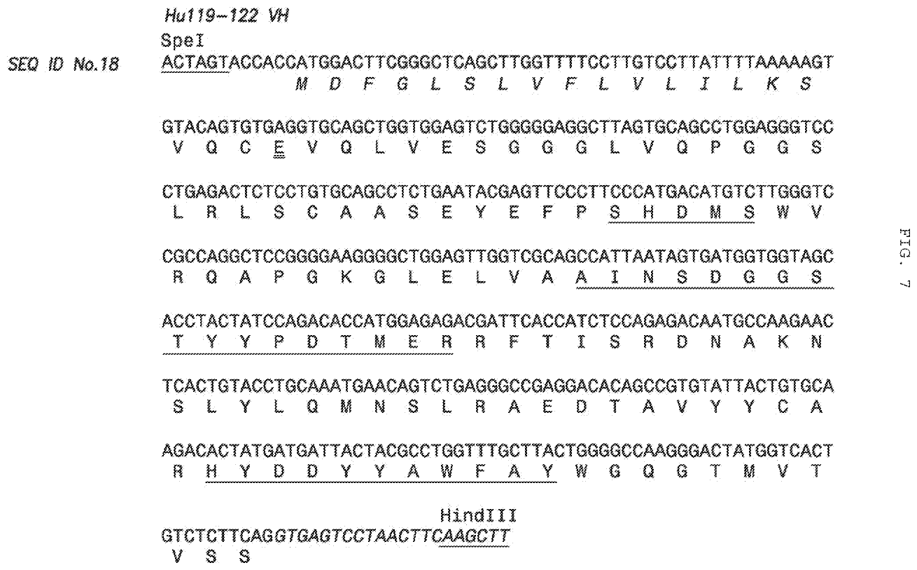

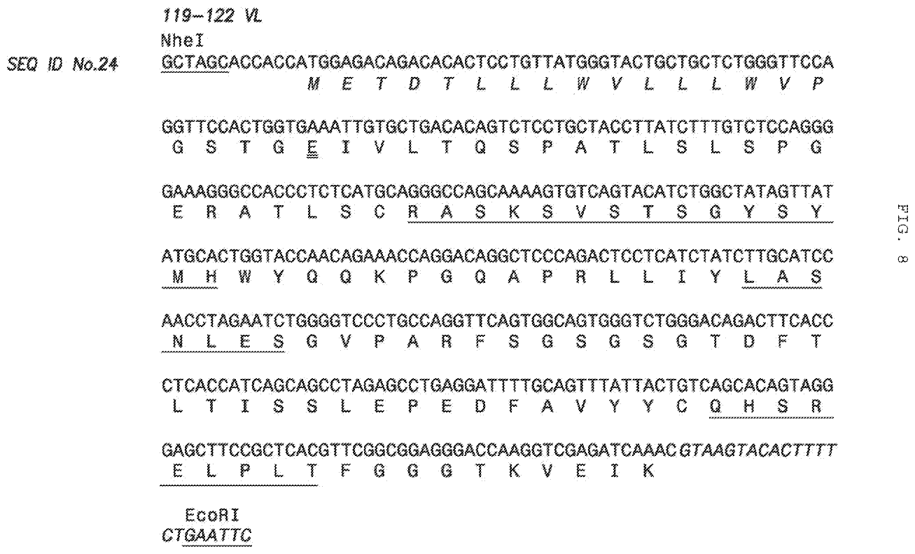

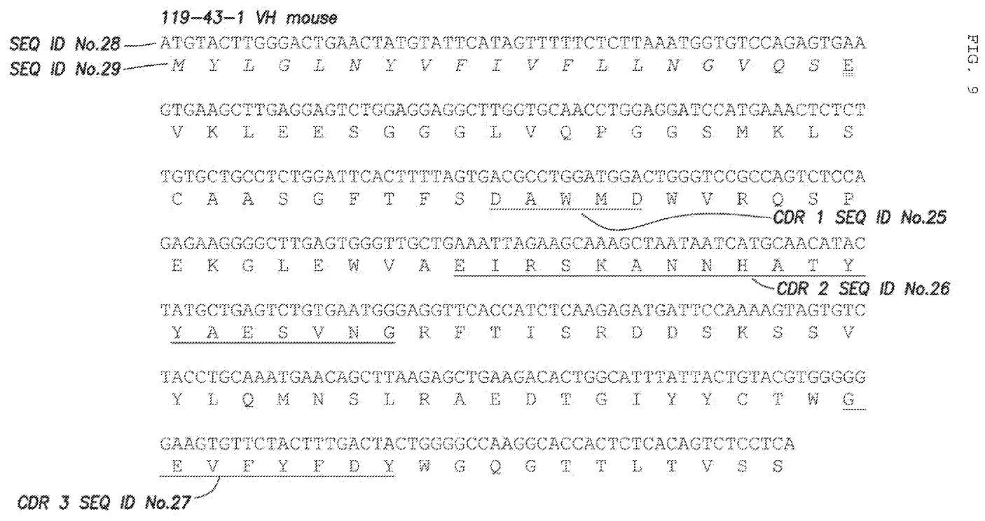

FIGS. 1-12 show sequences of the anti-OX40 ABPs of a combination of the invention, or a method or use thereof, e.g., CDRs and VH and VL sequences of the ABPs. In another embodiment, the anti-OX40 ABP of a combination of the invention, or a method or use thereof, comprises one or more of the CDRs or VH or VL sequences, or sequences with at least 90% (e.g., 90%, 91%, 92%, 93%, 94%, 95%, 96%, 97%, 98%, 99%, or 100%) sequence identity thereto, shown in the Figures herein. FIG. 1 includes a disclosure of residues 1-30, 36-49, 67-98, and 121-131 of SEQ ID NO:70. X61012 is disclosed as SEQ ID NO: 70. FIG. 2 includes a disclosure of residues 1-23, 35-49, 57-88, and 102-111 of SEQ ID NO:71. A3388641 is disclosed as SEQ ID NO:71. FIG. 3 includes a disclosure of the amino acid sequence as SEQ ID NO:72. FIG. 4 includes a disclosure of the amino acid sequence as SEQ ID NO:73. FIG. 5 includes a disclosure of residues 17-46, 52-65, 83-114, and 126-136 of SEQ ID NO:74. Z14189 is disclosed as SEQ ID NO:74. FIG. 6 includes a disclosure of residues 21-43, 55-69, 77-108, and 118-127 of SEQ ID NO:75. M29469 is disclosed as SEQ ID NO:75. FIG. 7 includes a disclosure of the amino acid sequence as SEQ ID NO:76. FIG. 8 includes a disclosure of the amino acid sequence as SEQ ID NO:77.

FIG. 1 shows the alignment of the amino acid sequences of murine 106-222, humanized 106-222 (Hu106), and human acceptor X61012 (GenBank accession number) VH sequences. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). In FIG. 1, CDR sequences defined by Kabat et al. (1991) are underlined in 106-222 VH. CDR residues in X61012 VH are omitted in the figure. Human VH sequences homologous to the 106-222 VH frameworks were searched for within the GenBank database, and the VH sequence encoded by the human X61012 cDNA (X61012 VH) was chosen as an acceptor for humanization. The CDR sequences of 106-222 VH were first transferred to the corresponding positions of X61012 VH. Next, at framework positions where the three-dimensional model of the 106-222 variable regions suggested significant contact with the CDRs, amino acid residues of mouse 106-222 VH were substituted for the corresponding human residues. These substitutions were performed at positions 46 and 94 (underlined in Hu106 VH). In addition, a human framework residue that was found to be atypical in the corresponding V region subgroup was substituted with the most typical residue to reduce potential immunogenicity. This substitution was performed at position 105 (double-underlined in Hu106 VH).

FIG. 2 shows alignment of the amino acid sequences of murine 106-222, humanized 106-222 (Hu106), and human acceptor AJ388641 (GenBank accession number) VL sequences. Amino acid residues are shown in single letter code. Numbers above the sequences indicate the locations according to Kabat et al. (1991). CDR sequences defined by Kabat et al. are underlined in 106-222 VH. CDR residues in AJ388641 VL are omitted in the figure. Human VL sequences homologous to the 106-222 VL frameworks were searched for within the GenBank database, and the VL sequence encoded by the human AJ388641 cDNA (AJ388641 VL) was chosen as an acceptor for humanization. The CDR sequences of 106-222 VL were transferred to the corresponding positions of A3388641 VL. No framework substitutions were performed in the humanized form.

FIG. 3 shows the nucleotide sequence of the Hu106 VH gene flanked by SpeI and HindIII sites (underlined) along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The intron sequence is in italic.

FIG. 4 shows the nucleotide sequence of the Hu106-222 VL gene flanked by Nhel and EcoRI sites (underlined) along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

FIG. 5 shows the alignment of the amino acid sequences of 119-122, humanized 119-122 (Hu119), and human acceptor Z14189 (GenBank accession number) VH sequences. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). CDR sequences defined by Kabat et al. (1991) are underlined in 119-122 VH. CDR residues in Z14189 VH are omitted in the figure. Human VH sequences homologous to the 119-122 VH frameworks were searched for within the GenBank database, and the VH sequence encoded by the human Z14189 cDNA (Z14189 VH) was chosen as an acceptor for humanization. The CDR sequences of 119-122 VH were first transferred to the corresponding positions of Z14189 VH. Next, at framework positions where the three-dimensional model of the 119-122 variable regions suggested significant contact with the CDRs, amino acid residues of mouse 119-122 VH were substituted for the corresponding human residues. These substitutions were performed at positions 26, 27, 28, 30 and 47 (underlined in the Hu119 VH sequence) as shown on the figure.

FIG. 6 shows the alignment of the amino acid sequences of 119-122, humanized 119-122 (Hu119), and human acceptor M29469 (GenBank accession number) VL sequences. Amino acid residues are shown in single letter code. Numbers above the sequences indicate the locations according to Kabat et al. (1991). CDR sequences defined by Kabat et al. (1) are underlined in 119-122 VL. CDR residues in M29469 VL are omitted in the sequence. Human VL sequences homologous to the 119-122 VL frameworks were searched for within the GenBank database, and the VL sequence encoded by the human M29469 cDNA (M29469 VL) was chosen as an acceptor for humanization. The CDR sequences of 119-122 VL were transferred to the corresponding positions of M29469 VL. No framework substitutions were needed in the humanized form.

FIG. 7 shows the nucleotide sequence of the Hu119 VH gene flanked by SpeI and HindIII sites (underlined) along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic.

FIG. 8 shows the nucleotide sequence of the Hu119 VL gene flanked by Nhel and EcoRI sites (underlined) along with the deduced amino acid sequence. Amino acid residues are shown in single letter code. The signal peptide sequence is in italic. The N-terminal amino acid residue (E) of the mature VL is double-underlined. CDR sequences according to the definition of Kabat et al. (1991) are underlined. The intron sequence is in italic.

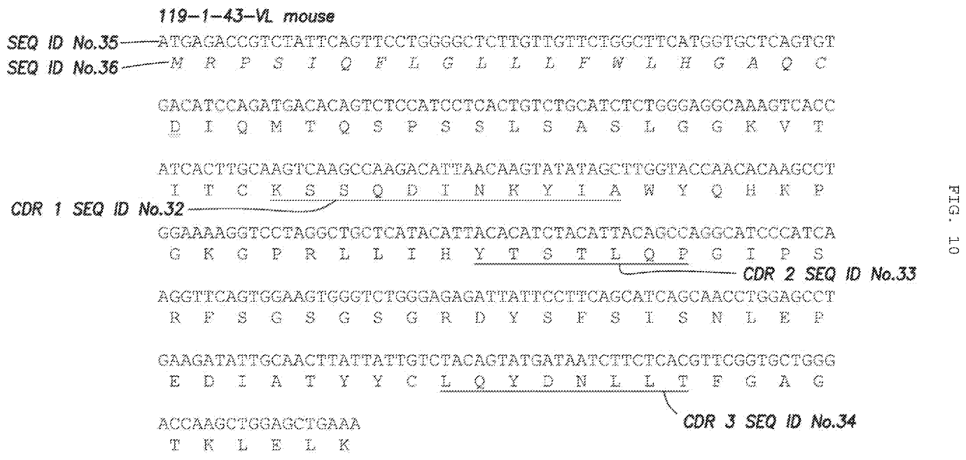

FIG. 9 shows the nucleotide sequence of mouse 119-43-1 VH cDNA along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (Sequences of Proteins of Immunological Interests, Fifth edition, NIH Publication No. 91-3242, U.S. Department of Health and Human Services, 1991) are underlined.

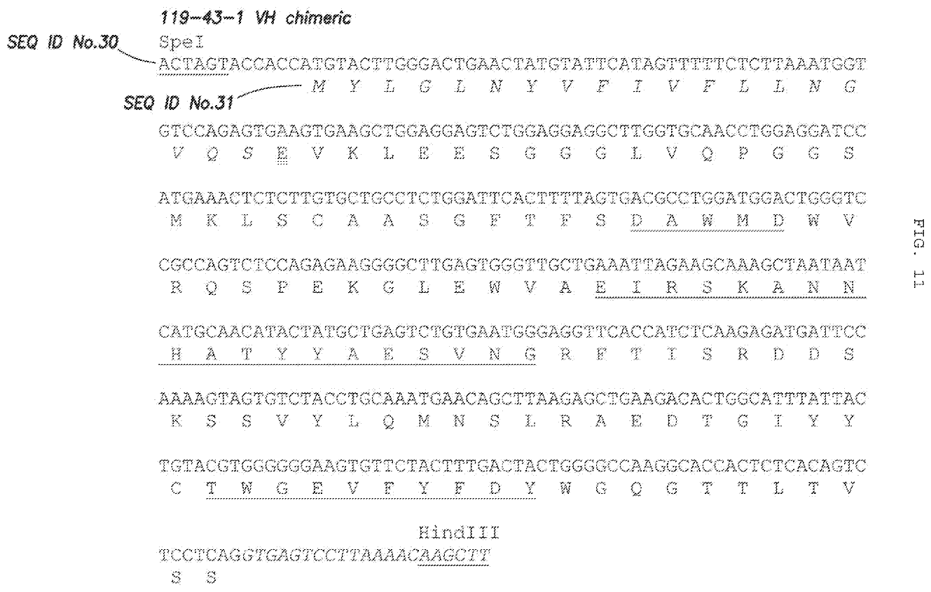

FIG. 10 shows the nucleotide sequence of mouse 119-43-1 VL cDNA along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined.

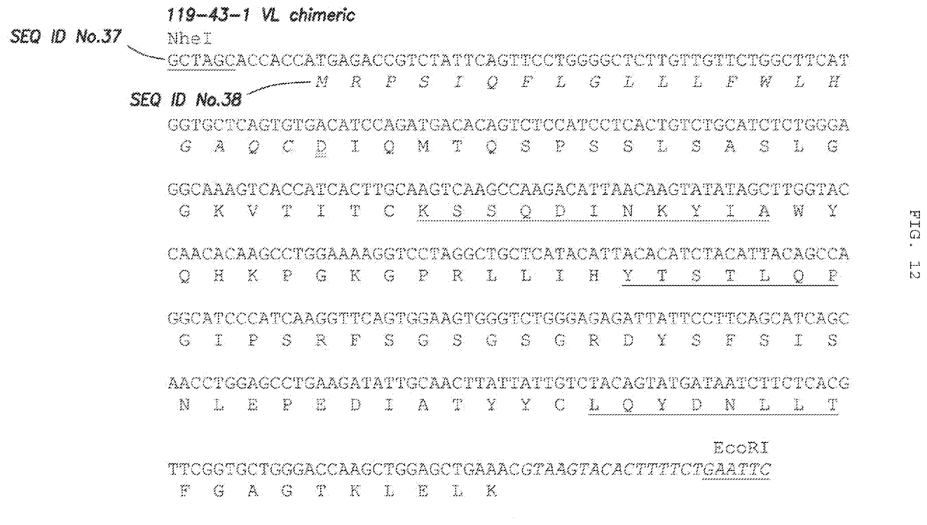

FIG. 11 shows the nucleotide sequence of the designed 119-43-1 VH gene flanked by SpeI and HindIII sites (underlined) along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic.

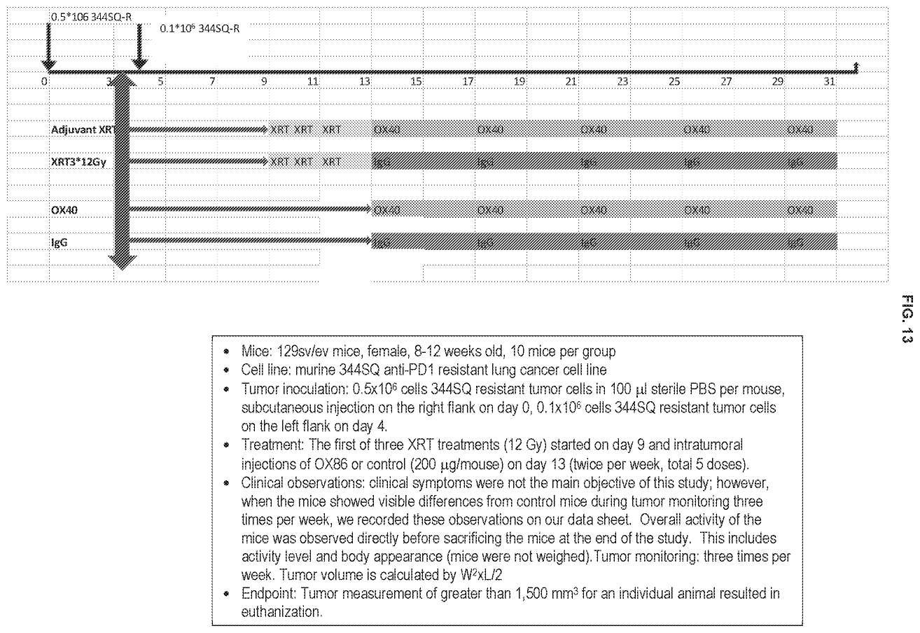

FIG. 12 shows the nucleotide sequence of the designed 119-43-1 VL gene flanked by Nhel and EcoRI sites (underlined) along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

In one embodiment, the anti-OX40 ABP of a combination of the invention, or a method or use thereof, comprises the CDRs of the 106-222 antibody, e.g., CDRH1, CDRH2, and CDRH3 having the amino acid sequence as set forth in SEQ ID NOS:1, 2, and 3, and e.g., CDRL1, CDRL2, and CDRL3 having the sequences as set forth in SEQ ID NOS:7, 8, and 9 respectively. In one embodiment, the ABP of a combination of the invention, or a method or use thereof, comprises the CDRs of the 106-222, Hu106 or Hu106-222 antibody as disclosed in WO2012/027328 (PCT/US2011/048752), international filing date 23 Aug. 2011.

As described herein, ANTIBODY 106-222 is a humanized monoclonal antibody that binds to human OX40 as disclosed in WO2012/027328 and described herein as an antibody comprising CDRH1, CDRH2, and CDRH3 having the amino acid sequence as set forth in SEQ ID NOS:1, 2, and 3, and e.g., CDRL1, CDRL2, and CDRL3 having the sequences as set forth in SEQ ID NOS:7, 8, and 9, respectively and an antibody comprising VH having an amino acid sequence as set forth in SEQ ID NO:5 and a VL having an amino acid sequence as set forth in SEQ ID NO:11.

In a further embodiment, the anti-OX40 ABP of a combination of the invention, or a method or use thereof, comprises the VH and VL regions of the 106-222 antibody as shown in FIG. 6 and FIG. 7 herein, e.g., a VH having an amino acid sequence as set forth in SEQ ID NO:4 and a VL having an amino acid sequence as set forth in SEQ ID NO:10. In another embodiment, the ABP of a combination of the invention, or a method or use thereof, comprises a VH having an amino acid sequence as set forth in SEQ ID NO:5, and a VL having an amino acid sequence as set forth in SEQ ID NO:11. In a further embodiment, the anti-OX40 ABP of a combination of the invention, or a method or use thereof, comprises the VH and VL regions of the 106-222 antibody or the Hu106 antibody as disclosed in WO2012/027328 (PCT/US2011/048752), international filing date 23 Aug. 2011. In a further embodiment, the anti-OX40 ABP of a combination of the invention, or a method or use thereof, is 106-222, Hu106-222 or Hu106, e.g., as disclosed in WO2012/027328 (PCT/US2011/048752), international filing date 23 Aug. 2011. In a further embodiment, the ABP of a combination of the invention, or a method or use thereof, comprises CDRs or VH or VL or antibody sequences with at least 90% (e.g., 90%, 91%, 92%, 93%, 94%, 95%, 96%, 97%, 98%, 99%, or 100%) sequence identity to the sequences in this paragraph.

In another embodiment, the anti-OX40 ABP of a combination of the invention, or a method or use thereof, comprises the CDRs of the 119-122 antibody, e.g., CDRH1, CDRH2, and CDRH3 having the amino acid sequence as set forth in SEQ ID NOs:13, 14, and 15 respectively. In another embodiment, the anti-OX40 ABP of a combination of the invention, or a method or use thereof, comprises the CDRs of the murine 119-122 or Hu119 or Hu119-222 antibody as disclosed in WO2012/027328 (PCT/US2011/048752), international filing date 23 Aug. 2011. In a further embodiment, the anti-OX40 ABP of a combination of the invention, or a method or use thereof, comprises a VH having an amino acid sequence as set forth in SEQ ID NO:16, and a VL having the amino acid sequence as set forth in SEQ ID NO:22. In another embodiment, the anti-OX40 ABP of a combination of the invention, or a method or use thereof, comprises a VH having an amino acid sequence as set forth in SEQ ID NO:17 and a VL having the amino acid sequence as set forth in SEQ ID NO:23. In a further embodiment, the anti-OX40 ABP of a combination of the invention, or a method or use thereof, comprises the VH and VL regions of the murine 119-122 or Hu119 or Hu119-222 antibody as disclosed in WO2012/027328 (PCT/US2011/048752), international filing date 23 Aug. 2011. In a further embodiment, the ABP of a combination of the invention, or a method or use thereof, is murine 119-222 or Hu119 or Hu119-222 antibody, e.g., as disclosed in WO2012/027328 (PCT/US2011/048752), international filing date 23 Aug. 2011. In a further embodiment, the ABP comprises CDRs or VH or VL or antibody sequences with at least 90% (e.g., 90%, 91%, 92%, 93%, 94%, 95%, 96%, 97%, 98%, 99%, or 100%) sequence identity to the sequences in this paragraph.

In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, comprises one of the VH and one of the VL regions of the 119-43-1 antibody. In a further embodiment, the anti-OX40 ABP of a combination of the invention, or a method or use thereof, comprises the VH and VL regions of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, is murine 119-43-1 or 119-43-1 chimeric. In a further embodiment, the anti-OX40 ABP of a combination of the invention, or a method or use thereof, as disclosed in WO2013/028231 (PCT/US2012/024570), international filing date 9 Feb. 2012. In further embodiments, any one of the anti-OX40 ABPs described in this paragraph are humanized. In further embodiments, any one of the any one of the ABPs described in this paragraph are engineered to make a humanized antibody. In a further embodiment, the anti-OX40 ABP of a combination of the invention, or a method or use thereof, comprises CDRs or VH or VL or antibody sequences with at least 90% (e.g., 90%, 91%, 92%, 93%, 94%, 95%, 96%, 97%, 98%, 99%, or 100%) sequence identity to the sequences in this paragraph.

In another embodiment, further embodiment, any mouse or chimeric sequences of any anti-OX40 ABP of a combination of the invention, or a method or use thereof, are engineered to make a humanized antibody.

In one embodiment, the anti-OX40 ABP of a combination of the invention, or a method or use thereof, comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In another embodiment, the anti-OX40 ABP of a combination of the invention, or a method or use thereof, comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO: 15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In another embodiment, the anti-OX40 ABP of a combination of the invention, or a method or use thereof, comprises: a heavy chain variable region CDR1 comprising the amino acid sequence of SEQ ID NO:1 or 13; a heavy chain variable region CDR2 comprising the amino acid sequence of SEQ ID NO:2 or 14; and/or a heavy chain variable region CDR3 comprising the amino acid sequence of SEQ ID NO:3 or 15, or a heavy chain variable region CDR having 90 percent identity thereto.

In another embodiment, the anti-OX40 ABP of a combination of the invention, or a method or use thereof, comprises: a light chain variable region CDR1 comprising the amino acid sequence of SEQ ID NO:7 or 19; a light chain variable region CDR2 comprising the amino acid sequence of SEQ ID NO:8 or 20 and/or a light chain variable region CDR3 comprising the amino acid sequence of SEQ ID NO:9 or 21, or a heavy chain variable region having 90 percent identity thereto.

In another embodiment, the anti-OX40 ABP of a combination of the invention, or a method or use thereof, comprises: a light chain variable region (“VL”) comprising the amino acid sequence of SEQ ID NO:10, 11, 22 or 23, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:10, 11, 22 or 23. In another embodiment, the anti-OX40 ABP of a combination of the invention, or a method or use thereof, comprises a heavy chain variable region (“VH”) comprising the amino acid sequence of SEQ ID NO:4, 5, 16 or 17, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:4, 5, 16 or 17. In another embodiment, the anti-OX40 ABP of a combination of the invention, or a method or use thereof, comprises a variable heavy sequence of SEQ ID NO:5 and a variable light sequence of SEQ ID NO:11, or a sequence having 90 (e.g., 90%, 91%, 92%, 93%, 94%, 95%, 96%, 97%, 98%, 99%, or 100%) percent sequence identity thereto. In another embodiment, the anti-OX40 ABP of a combination of the invention, or a method or use thereof, comprises a variable heavy sequence of SEQ ID NO:17 and a variable light sequence of SEQ ID NO:23 or a sequence having 90 (e.g., 90%, 91%, 92%, 93%, 94%, 95%, 96%, 97%, 98%, 99%, or 100%) percent sequence identity thereto.

In another embodiment, the anti-OX40 ABP of a combination of the invention, or a method or use thereof, comprises a variable light chain encoded by the nucleic acid sequence of SEQ ID NO:12, or 24, or a nucleic acid sequence with at least 90% (e.g., 90%, 91%, 92%, 93%, 94%, 95%, 96%, 97%, 98%, 99%, or 100%) sequence identity to the nucleotide sequences of SEQ ID NO:12 or 24. In another embodiment, the anti-OX40 ABP of a combination of the invention, or a method or use thereof, comprises a variable heavy chain encoded by a nucleic acid sequence of SEQ ID NO:6 or 18, or a nucleic acid sequence with at least 90% (e.g., 90%, 91%, 92%, 93%, 94%, 95%, 96%, 97%, 98%, 99%, or 100%) sequence identity to nucleotide sequences of SEQ ID NO:6 or 18.

Also provided herein are monoclonal antibodies. In one embodiment, the monoclonal antibodies comprise a variable light chain comprising the amino acid sequence of SEQ ID NO:10 or 22, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:10 or 22. Further provided are monoclonal antibodies comprising a variable heavy chain comprising the amino acid sequence of SEQ ID NO:4 or 16, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:4 or 16.

In one embodiment, the monoclonal antibodies comprise a variable light chain comprising the amino acid sequence of SEQ ID NO:11 or 23, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:11 or 23. Further provided are monoclonal antibodies comprising a variable heavy chain comprising the amino acid sequence of SEQ ID NO:5 or 17, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:5 or 17.

In one embodiment, the monoclonal antibodies comprise a variable light chain comprising the amino acid sequence of SEQ ID NO:11, or an amino acid sequence with at least 90% (e.g., 90%, 91%, 92%, 93%, 94%, 95%, 96%, 97%, 98%, 99%, or 100%) sequence identity to the amino acid sequence of SEQ ID NO:11. Further provided are monoclonal antibodies comprising a variable heavy chain comprising the amino acid sequence of SEQ ID NO:5, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:5.

Further provided are monoclonal antibodies comprising a variable light chain comprising the amino acid sequence of SEQ ID NO:11, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:11, and a variable heavy chain comprising the amino acid sequence of SEQ ID NO:5, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:5.

In one embodiment, the monoclonal antibodies comprise a light chain comprising the amino acid sequence of SEQ ID NO:49, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:49. Further provided are monoclonal antibodies comprising a heavy chain comprising the amino acid sequence of SEQ ID NO:48, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:48.

Further provided are monoclonal antibodies comprising a light chain comprising the amino acid sequence of SEQ ID NO:49, or an amino acid sequence with at least 90% (e.g., 90%, 91%, 92%, 93%, 94%, 95%, 96%, 97%, 98%, 99%, or 100%) sequence identity to the amino acid sequence of SEQ ID NO:49, and a heavy chain comprising the amino acid sequence of SEQ ID NO:48, or an amino acid sequence with at least 90% (e.g., 90%, 91%, 92%, 93%, 94%, 95%, 96%, 97%, 98%, 99%, or 100%) sequence identity to the amino acid sequences of SEQ ID NO:48.

Heavy Chain of ANTIBODY 106-222:
(SEQ ID NO: 48)
QVQLVQSGSELKKPGASVKVSCKASGYTFTDYSMHWVRQAPGQGLKWMGWINTETGE
PTYADDFKGRFVFSLDTSVSTAYLQISSLKAEDTAVYYCANPYYDYVSYYAMDYWGQGT
TVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK
Light Chain of ANTIBODY 106-222:
(SEQ ID NO: 49)
DIQMTQSPSSLSASVGDRVTITCKASQDVSTAVAWYQQKPGKAPKWYSASYLYTGVPS
RFSGSGSGTDFTFTISSLQPEDIATYYCQQHYSTPRTFGQGTKLEIKRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC
Heavy Chain Variable Region of ANTIBODY 106-222:
(SEQ ID NO: 5)
QVQLVQSGSELKKPGASVKVSCKASGYTFTDYSMHVVVRQAPGQGLKWMGWINTETGE
PTYADDFKGRFVFSLDTSVSTAYLQISSLKAEDTAVYYCANPYYDYVSYYAMDYWGQGT
TVTVSS
Light Chain Variable Region of ANTIBODY 106-222:
(SEQ ID NO: 11)
DIQMTQSPSSLSASVGDRVTITCKASQDVSTAVAWYQQKPGKAPKWYSASYLYTGVPS
RFSGSGSGTDFTFTISSLQPEDIATYYCQQHYSTPRTFGQGTKLEIK
CDR sequences of ANTIBODY 106-222:
HC CDR1:
(SEQ ID NO: 1)
Asp Tyr Ser Met His
HC CDR2:
(SEQ ID NO: 2)
Trp Ile Asn Thr Glu Thr Gly Glu Pro Thr Tyr Ala Asp Asp Phe Lys Gly
HC CDR3:
(SEQ ID NO: 3)
Pro Tyr Tyr Asp Tyr Val Ser Tyr Tyr Ala Met Asp Tyr
LC CDR1:
(SEQ ID NO: 7)
Lys Ala Ser Gln Asp Val Ser Thr Ala Val Ala
LC CDR2:
(SEQ ID NO: 8)
Ser Ala Ser Tyr Leu Tyr Thr
LC CDR3:
(SEQ ID NO: 9)
Gln Gln His Tyr Ser Thr Pro Arg Thr

PD-1 Antigen Binding Proteins

The combinations, and methods and uses thereof, of the invention may also comprise anti-PD-1 antigen binding proteins that bind PD-1 (such as human PD-1), such as antagonists molecules (such as antibodies) that block binding with a PD-1 ligand such as PD-L1 or PD-L2.

ABPs that bind human PD-1 receptor are provided herein (i.e. an anti-PD-1 ABP, sometimes referred to herein as an “anti-PD-1 ABP” such as an “anti-PD-1 antibody”). These ABPs such as antibodies are useful in the treatment or prevention of acute or chronic diseases or conditions whose pathology involves PD-1 signalling. In one aspect, an antigen binding protein, or isolated human antibody or functional fragment of such protein or antibody, that binds to human PD-1 and is effective as a cancer treatment or treatment against disease is described, for example in combination with another compound such as an anti-OX40 ABP, suitably an agonist anti-OX40 ABP. Any of the antigen binding proteins or antibodies disclosed herein may be used as a medicament. Any one or more of the antigen binding proteins or antibodies may be used in the methods or compositions to treat a cancer, e.g., one disclosed herein.

The isolated ABPs such as antibodies as described herein bind to human PD-1, and may bind to human PD-1 encoded by the gene Pdcd1, or genes or cDNA sequences having 90 percent homology or 90 percent identity thereto. The complete hPD-1 mRNA sequence can be found under GenBank Accession No. U64863. The protein sequence for human PD-1 can be found at GenBank Accession No. AAC51773.

Antigen binding proteins and antibodies that bind and/or modulate PD-1 are known in the art. Exemplary anti-PD-1 ABPs of a combination of the invention, or a method or use thereof, are disclosed, for example in U.S. Pat. Nos. 8,354,509; 8,900,587; 8,008,449, each of which is incorporated by reference in its entirety herein (To the extent any definitions conflict, this instant application controls). PD-1 antibodies and methods of using in treatment of disease are described in U.S. Pat. Nos. 7,595,048; 8,168,179; 8,728,474; 7,722,868; 8,008,449; 7,488,802; 7,521,051; 8,088,905; 8,168,757; 8,354,509; and US Publication Nos. US20110171220; US20110171215; and US20110271358. Combinations of CTLA-4 and PD-1 antibodies are described in U.S. Pat. No. 9,084,776.

In another embodiment, any mouse or chimeric sequences of any anti-PD-1 ABP of a combination of the invention, or a method or use thereof, are engineered to make a humanized antibody.

In another embodiment, the anti-PD-1 ABP of a combination of the invention, or a method or use thereof, comprises one or more (e.g., all) of the CDRs (SEQ ID NOS:54-59) or VH (SEQ ID NO:52) or VL (SEQ ID NO:53) or HC (heavy chain) (SEQ ID NO:50) or LC (light chain) (SEQ ID NO:51) sequences of pembrolizumab, or sequences with at least 90% (e.g., 90, 91, 92, 93, 94, 95, 96, 97, 98, 99, or 100%) identity thereto.

The HC and LC CDRs of permolizumab are provided below. In one embodiment, the anti-PD-1 ABP of a combination of the invention, or a method or use thereof, comprises: (a) a heavy chain variable region CDR1 (SEQ ID NO:54) of pembrolizumab; (b) a heavy chain variable region CDR2 (SEQ ID NO:55) of pembrolizumab; (c) a heavy chain variable region CDR3 (SEQ ID NO:56) of pembrolizumab; (d) a light chain variable region CDR1 (SEQ ID NO:57) of pembrolizumab; (e) a light chain variable region CDR2 (SEQ ID NO:58) of pembrolizumab; and (f) a light chain variable region CDR3 (SEQ ID NO:59) of pembrolizumab.

In another embodiment, the anti-PD-1 of a combination of the invention, or a method or use thereof, comprises: a heavy chain variable region CDR1 (SEQ ID NO:54) of pembrolizumab; a heavy chain variable region CDR2 (SEQ ID NO:55) of pembrolizumab and/or a heavy chain variable region CDR3 (SEQ ID NO:56) of pembrolizumab.

In another embodiment, the anti-PD-1 of a combination of the invention, or a method or use thereof, comprises: a light chain variable region CDR1 (SEQ ID NO:57) of pembrolizumab; a light chain variable region CDR2 (SEQ ID NO:58) of pembrolizumab and/or a light chain variable region CDR3 (SEQ ID NO:59) of pembrolizumab.

In another embodiment, the anti-PD-1 ABP of a combination of the invention, or a method or use thereof, comprises: a light chain variable region (“VL”) (SEQ ID NO:53) of pembrolizumab, or an amino acid sequence with at least 90% (e.g., 90, 91, 92, 93, 94, 95, 96, 97, 98, 99, or 100%) identity to the amino acid sequence of the VL of pembrolizumab.

In another embodiment, the anti-PD-1 ABP of a combination of the invention, or a method or use thereof, comprises a heavy chain variable region (“VH”) (SEQ ID NO:52) of pembrolizumab, or an amino acid sequence with at least 90% (e.g., 90, 91, 92, 93, 94, 95, 96, 97, 98, 99, or 100%) identity to the amino acid sequence of the VH of pembrolizumab.

In another embodiment, the anti-PD-1 ABP of a combination of the invention, or a method or use thereof, comprises: a light chain variable region (“VL”) of pembrolizumab, or an amino acid sequence with at least 90% (e.g., 90, 91, 92, 93, 94, 95, 96, 97, 98, 99, or 100%) identity to the amino acid sequence of the VL of pembrolizumab and the anti-PD-1 ABP of a combination of the invention, or a method or use thereof, comprises a heavy chain variable region (“VH”) of pembrolizumab, or an amino acid sequence with at least 90% (e.g., 90, 91, 92, 93, 94, 95, 96, 97, 98, 99, or 100%) identity to the amino acid sequence of the VH of pembrolizumab.

In another embodiment, the anti-PD-1 ABP of a combination of the invention, or a method or use thereof, comprises: a light chain (“LC”) (SEQ ID NO:51) of pembrolizumab, or an amino acid sequence with at least 90% (e.g., 90, 91, 92, 93, 94, 95, 96, 97, 98, 99, or 100%) identity to the amino acid sequence of the LC of pembrolizumab.

In another embodiment, the anti-PD-1 ABP of a combination of the invention, or a method or use thereof, comprises a heavy chain (“HC”) (SEQ ID NO:50) of pembrolizumab, or an amino acid sequence with at least 90% (e.g., 90, 91, 92, 93, 94, 95, 96, 97, 98, 99, or 100%) identity to the amino acid sequence of the HC of pembrolizumab.

In another embodiment, the anti-PD-1 ABP of a combination of the invention, or a method or use thereof, comprises: a light chain (“LC”) (SEQ ID NO:51) of pembrolizumab, or an amino acid sequence with at least 90% (e.g., 90, 91, 92, 93, 94, 95, 96, 97, 98, 99, or 100% identity to the amino acid sequence of the LC of pembrolizumab and the anti-PD-1 ABP of a combination of the invention, or a method or use thereof, comprises a heavy chain (“HC”) (SEQ ID NO:50) of pembrolizumab, or an amino acid sequence with at least 90% (e.g., 90, 91, 92, 93, 94, 95, 96, 97, 98, 99, or 100%) identity to the amino acid sequence of the HC of pembrolizumab.

An anti-OX40 ABP (e.g., an agonist ABP, e.g., an anti-hOX40 ABP, e.g., antibody), e.g., an antibody described herein, can be used in combination with an ABP (e.g., antagonist ABP, e.g antagonist antibody) against PD-1 (e.g., human PD-1). For example, an anti-OX40 antibody can be used in combination with pembrolizumab.

While in development, pembrolizumab (KEYTRUDA®) was known as MK3475 and as lambrolizumab. Pembrolizumab (KEYTRUDA®) is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. The recommended dose of pembrolizumab is 2 mg/kg administered as an intravenous infusion over 30 minutes every 3 weeks until disease progression or unacceptable toxicity.

Pembrolizumab is a humanized monoclonal antibody that blocks the interaction between PD-1 and its ligands, PD-L1 and PD-L2. Pembrolizumab is an IgG4 kappa immunoglobulin with an approximate molecular weight of 149 kDa.

Pembrolizumab for injection is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate-80 (0.4 mg), sucrose (140 mg). May contain hydrochloric acid/sodium hydroxide to adjust pH to 5.5.

Pembrolizumab injection is a sterile, preservative-free, clear to slightly opalescent, colorless to slightly yellow solution that requires dilution for intravenous infusion. Each vial contains 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and Water for Injection, USP.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Pembrolizumab is a monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Pembrolizumab is described, e.g., in U.S. Pat. Nos. 8,354,509 and 8,900,587.

The approved product is pembrolizumab (KEYTRUDA®) for injection, for intravenous infusion of the active ingredient pembrolizumab, available as a 50 mg lyophilized powder in a single-usevial for reconstitution. Pembrolizumab has been approved for the treatment of patients with unresectable or metastatic melanoma and disease progression following ipilimumab and, if BRAF V600 mutation positive, a BRAF inhibitor. Pembrolizumab (KEYTRUDA®) is a humanized monoclonal antibody that blocks the interaction between PD-I and its ligands, PD-LI and PD-L2. Pembrolizumab is an IgG4 kappa immunoglobulin with an approximate molecular weight of 149 kDa. The amino acid sequence for pembrolizumab is as follows, and is set forth using the same one-letter amino acid code nomenclature provided in the table at column 15 of the U.S. Pat. No. 8,354,509:

Heavy Chain of pembrolizumab:
(SEQ ID NO: 50)
QVQLVQSGVE VKKPGASVKV SCKASGYTFT NYYMYWVRQA PGQGLEWMGG 50
INPSNGGTNF NEKFKNRVTL TTDSSTTTAY MELKSLQFDD TAVYYCARRD 100
YRFDMGFDYW GQGTTVTVSS ASTKGPSVFP LAPCSRSTSE STAALGCLVK 150
DYFPEPVTVS WNSGALTSGV HTFPAVLQSS GLYSLSSVVT VPSSSLGTKT 200
YTCNVDHKPS NTKVDKRVES KYGPPCPPCP APEFLGGPSV FLFPPKPKDT 250
LMISRTPEVT CVVVDVSQED PEVQFNWYVD GVEVHNAKTK PREEQFNSTY 300
VVSVLTVLH QDWLNGKEYK CKVSNKGLPS SIEKTISKAK GQPREPQVYT 350
LPPSQEEMTK NQVSLTCLVK GFYPSDIAVE WESNGQPENN YKTTPPVLDS 400
DGSFFLYSRL TVDKSRWQEG NVFSCSVMHE ALHNHYTQKS LSLSLGK 447
Light Chain of pembrolizumab:
(SEQ ID NO: 51)
EIVLTQSPAT LSLSPGERAT LSCRASKGVS TSGYSYLHWY QQKPGQAPRL 50
LIYLASYLES GVPARFSGSG SGTDFTLTIS SLEPEDFAVY YCQHSRDLPL 100
TFGGGTKVEI KRTVAAPSVF IFPPSDEQLK SGTASVVCLL NNFYPREAKV 150
QWKVDNALQS GNSQESVTEQ DSKDSTYSLS STLTLSKADY EKHKVYACEV 200
THQGLSSPVT KSFNRGEC 218
Heavy Chain Variable Region of pembrolizumab:
(SEQ ID NO: 52)
QVQLVQSGVE VKKPGASVKV SCKASGYTFT NYYMYWVRQA PGQGLEWMGG 50
INPSNGGTNF NEKFKNRVTL TTDSSTTTAY MELKSLQFDD TAVYYCARRD 100
YRFDMGFDYW GQGTTVTVSS
Light Chain Variable Region of pembrolizumab:
(SEQ ID NO: 53)
EIVLTQSPAT LSLSPGERAT LSCRASKGVS TSGYSYLHWY QQKPGQAPRL 50
LIYLASYLES GVPARFSGSG SGTDFTLTIS SLEPEDFAVY YCQHSRDLPL 100
TFGGGTKVEI K
CDR sequences of pembrolizumab:
HC CDR1:
(SEQ ID NO: 54)
Asn Tyr Tyr Met Tyr
HC CDR2:
(SEQ ID NO: 55)
Gly Ile Asn Pro Ser Asn Gly Gly Thr Asn Phe Asn Glu Lys Phe Lys Asn
HC CDR3:
(SEQ ID NO: 56)
Arg Asp Tyr Arg Phe Asp Met Gly Phe Asp Tyr
LC CDR1:
(SEQ ID NO: 57)
Arg Ala Ser Lys Gly Val Ser Thr Ser Gly Tyr Ser Tyr Leu His
LC CDR2:
(SEQ ID NO: 58)
Leu Ala Ser Tyr Leu Glu Ser
LC CDR3:
(SEQ ID NO: 59)
Gln His Ser Arg Asp Leu Pro Leu Thr

As another example, an anti-OX40 antibody can be used in combination with nivolumab (OPDIVO®). Nivolumab (OPDIVO®) is a programmed death receptor-1 (PD-1) blocking antibody indicated for the treatment of patients with:

unresectable or metastatic melanoma and disease progression following ipilimumab and, if BRAF V600 mutation positive, a BRAF inhibitor. This indication is approved under accelerated approval based on tumor response rate and durability of response. Continued approval for this indication may be contingent upon verification and description of clinical benefit in the confirmatory trials.

metastatic squamous non-small cell lung cancer with progression on or after platinum-based chemotherapy.

The recommended dose of nivolumab (OPDIVO®) is 3 mg/kg administered as an intravenous infusion over 60 minutes every 2 weeks until disease progression or unacceptable toxicity.

Binding of the PD-1 ligands, PD-L1 and PD-L2, to the PD-1 receptor found on T cells, inhibits T-cell proliferation and cytokine production. Upregulation of PD-1 ligands occurs in some tumors and signaling through this pathway can contribute to inhibition of active T-cell immune surveillance of tumors.

Nivolumab is a human immunoglobulin G4 (IgG4) monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

U.S. Pat. No. 8,008,449 exemplifies seven anti-PD-1 HuMAbs: 17D8, 2D3, 4H1, 5C4 (also referred to herein as nivolumab or BMS-936558), 4A1 1, 7D3 and 5F4. See also U.S. Pat. No. 8,779,105. Any one of these antibodies, or the CDRs thereof (or an amino acid sequence with at least 90% (e.g., 90, 91, 92, 93, 94, 95, 96, 97, 98, or 99%) identity to any of these amino acid sequences), can be used in the compositions and methods described herein.

```
Heavy Chain of nivolumab:
(SEQ ID NO: 98)
QVQLVESGGGVVQPGRSLRLDCKASGITESNSGMHWVRQAPGKGLEWVAVIWYDGSKRYYADSVKGRFTISRD
NSKNTLFLQMNSLRAEDTAVYYCATNDDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHNHYTQKSLSLSL
GK
Light Chain of nivolumab:
(SEQ ID NO: 99)
EIVLTQSPATLSLSPGERATLSCRASQSVSSYLAWYQQKPGQAPRLLIYDASNRATGIPARFSGSGSGTDFTL
TISSLEPEDFAVYYCQQSSNWPRTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
Heavy Chain Variable region of nivolumab:
(SEQ ID NO: 100)
Gln Val Gln Leu Val Glu Ser Gly Gly Gly Val Val Gln Pro Gly Arg
1 5 10 15
Ser Leu Arg Leu Asp Cys Lys Ala Ser Gly Ile Thr Phe Ser Asn Ser
                        20 25 30
Gly Met His Trp Val Arg Gln Ala Pro Gly Lys Gly Leu Glu Trp Val
                35 40 45
Ala Val Ile Trp Tyr Asp Gly Ser Lys Arg Tyr Tyr Ala Asp Ser Val
        50 55 60
Lys Gly Arg Phe Thr Ile Ser Arg Asp Asn Ser Lys Asn Thr Leu Phe
65 70 75 80
Leu Gln Met Asn Ser Leu Arg Ala Glu Asp Thr Ala Val Tyr Tyr Cys
                                85 90 95
Ala Thr Asn Asp Asp Tyr Trp Gly Gln Gly Thr Leu Val Thr Val Ser
                        100 105 110
Ser
Light Chain Variable region of nivolumab:
(SEQ ID NO: 101)
Glu Ile Val Leu Thr Gln Ser Pro Ala Thr Leu Ser Leu Ser Pro Gly
1 5 10 15
Glu Arg Ala Thr Leu Ser Cys Arg Ala Ser Gln Ser Val Ser Ser Tyr
                        20 25 30
Leu Ala Trp Tyr Gln Gln Lys Pro Gly Gln Ala Pro Arg Leu Leu Ile
                35 40 45
Tyr Asp Ala Ser Asn Arg Ala Thr Gly Ile Pro Ala Arg Phe Ser Gly
        50 55 60
Ser Gly Ser Gly Thr Asp Phe Thr Leu Thr Ile Ser Ser Leu Glu Pro
65 70 75 80
Glu Asp Phe Ala Val Tyr Tyr Cys Gln Gln Ser Ser Asn Trp Pro Arg
                                85 90 95
Thr Phe Gly Gln Gly Thr Lys Val Glu Ile Lys
                        100 105
CDR sequences of nivolumab:
HC CDR1:
(SEQ ID NO: 102)
Asn Ser Gly Met His
HC CDR2:
(SEQ ID NO: 103)
Val Ile Trp Tyr Asp Gly Ser Lys Arg Tyr Tyr Ala Asp Ser Val Lys Gly
HC CDR3:
(SEQ ID NO: 104)
Asn Asp Asp Tyr
LC CDR1:
(SEQ ID NO: 105)
Arg Ala Ser Gln Ser Val Ser Ser Tyr Leu Ala
LC CDR2:
(SEQ ID NO: 106)
Asp Ala Ser Asn Arg Ala Thr
LC CDR3:
(SEQ ID NO: 107)
Gln Gln Ser Ser Asn Trp Pro Arg Thr
```

In another embodiment, the anti-PD-1 ABP of a combination of the invention, or a method or use thereof, comprises one or more (e.g., all) of the CDRs (SEQ ID NOs:102-107) or VH (SEQ ID NO:100) or VL (SEQ ID NO:101) or HC (heavy chain) (SEQ ID NO:98) or LC (light chain) (SEQ ID NO:99) sequences of nivolumab, or sequences with at least 90% (e.g., 90%, 91%, 92%, 93%, 94%, 95%, 96%, 97%, 98%, 99%, or 100%) sequence identity thereto.

The HC and LC CDRs of nivolumab are provided herein. In one embodiment, the anti-PD-1 ABP of a combination of the invention, or a method or use thereof, comprises: (a) a heavy chain variable region CDR1 (SEQ ID NO:102) of nivolumab; (b) a heavy chain variable region CDR2 (SEQ ID NO:103) of nivolumab; (c) a heavy chain variable region CDR3 (SEQ ID NO:104) of nivolumab; (d) a light chain variable region CDR1 (SEQ ID NO:105) of nivolumab; (e) a light chain variable region CDR2 (SEQ ID NO:106) of nivolumab; and (f) a light chain variable region CDR3 (SEQ ID NO:107) of nivolumab.

In another embodiment, the anti-PD-1 of a combination of the invention, or a method or use thereof, comprises: a heavy chain variable region CDR1 (SEQ ID NO:102) of nivolumab; a heavy chain variable region CDR2 (SEQ ID NO:103) of nivolumab and/or a heavy chain variable region CDR3 (SEQ ID NO:104) of nivolumab.

In another embodiment, the anti-PD-1 of a combination of the invention, or a method or use thereof, comprises: a light chain variable region CDR1 (SEQ ID NO:105) of nivolumab; a light chain variable region CDR2 (SEQ ID NO:106) of nivolumab and/or a light chain variable region CDR3 (SEQ ID NO:107) of nivolumab.

In another embodiment, the anti-PD-1 ABP of a combination of the invention, or a method or use thereof, comprises: a light chain variable region (“VL”) (SEQ ID NO:101) of nivolumab, or an amino acid sequence with at least 90% (e.g., 90, 91, 92, 93, 94, 95, 96, 97, 98, 99, or 100%) identity to the amino acid sequence of the VL of nivolumab.

In another embodiment, the anti-PD-1 ABP of a combination of the invention, or a method or use thereof, comprises a heavy chain variable region (“VH”) (SEQ ID NO:100) of nivolumab, or an amino acid sequence with at least 90% (e.g., 90, 91, 92, 93, 94, 95, 96, 97, 98, 99, or 100%) identity to the amino acid sequence of the VH of nivolumab.

In another embodiment, the anti-PD-1 ABP of a combination of the invention, or a method or use thereof, comprises: a light chain variable region (“VL”) (SEQ ID NO:101) of nivolumab, or an amino acid sequence with at least 90% (e.g., 90, 91, 92, 93, 94, 95, 96, 97, 98, 99, or 100%) identity to the amino acid sequence of the VL of nivolumab and the anti-PD-1 ABP of a combination of the invention, or a method or use thereof, comprises a heavy chain variable region (“VH”) (SEQ ID NO:100) of nivolumab, or an amino acid sequence with at least 90% (e.g., 90, 91, 92, 93, 94, 95, 96, 97, 98, 99, or 100%) identity to the amino acid sequence of the VH of nivolumab.

In another embodiment, the anti-PD-1 ABP of a combination of the invention, or a method or use thereof, comprises: a light chain (“LC”) (SEQ ID NO:99) of nivolumab, or an amino acid sequence with at least 90% (e.g., 90, 91, 92, 93, 94, 95, 96, 97, 98, 99, or 100%) identity to the amino acid sequence of the LC of nivolumab.

In another embodiment, the anti-PD-1 ABP of a combination of the invention, or a method or use thereof, comprises a heavy chain (“HC”) (SEQ ID NO:98) of nivolumab, or an amino acid sequence with at least 90% (e.g., 90, 91, 92, 93, 94, 95, 96, 97, 98, 99, or 100%) identity to the amino acid sequence of the HC of nivolumab.

In another embodiment, the anti-PD-1 ABP of a combination of the invention, or a method or use thereof, comprises: a light chain (“LC”) (SEQ ID NO:99) of nivolumab, or an amino acid sequence with at least 90% (e.g., 90, 91, 92, 93, 94, 95, 96, 97, 98, 99, or 100%) identity to the amino acid sequence of the LC of nivolumab and the anti-PD-1 ABP of a combination of the invention, or a method or use thereof, comprises a heavy chain (“HC”) (SEQ ID NO:98) of nivolumab, or an amino acid sequence with at least 90% (e.g., 90, 91, 92, 93, 94, 95, 96, 97, 98, 99, or 100%) identity to the amino acid sequence of the HC of nivolumab.

An anti-OX40 ABP (e.g., an agonist ABP, e.g., an anti-hOX40 ABP, e.g., antibody), as described herein, can be used in combination with an ABP (e.g., antagonist ABP, e.g antagonist antibody) against PD-1 (e.g., human PD-1). For example, an anti-OX40 antibody can be used in combination with nivolumab.

In one aspect, the present invention provides methods of treating cancer in a mammal in need thereof comprising administering a therapeutically effective amount of an antigen binding protein that binds OX40 and an antigen binding protein that binds PD-1. In some embodiments, the method further includes radiotherapy. In some embodiments, the cancer is a solid tumor. The cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer. In another embodiment, the cancer is a liquid tumor.

In one embodiment, the antigen binding protein that binds OX40 and the antigen binding that binds PD-1 are administered at the same time. In another embodiment, antigen binding protein that binds OX40 and the antigen binding protein that binds PD-1 are administered sequentially, in any order. In one aspect, the antigen binding protein that binds OX40 and/or the antigen binding protein that binds PD-1 are administered systemically, e.g., intravenously. In another aspect, the antigen binding protein that binds OX40 and/or the antigen binding protein that binds PD-1 is administered intratumorally.

In one embodiment, the mammal is human.

Methods are provided wherein the tumor size of the cancer in said mammal is reduced by more than an additive amount compared with treatment with the antigen binding protein to OX40 and the antigen binding protein to PD-1 as used as a monotherapy. Suitably the combination may be synergistic.

In one embodiment, the antigen binding protein that binds OX40 binds to human OX40. In one embodiment, the antigen binding protein that binds to PD-1 binds to human PD-1. In one embodiment, the antigen binding protein that binds OX40 and/or the antigen binding protein that binds PD-1 is a humanized monoclonal antibody. In one embodiment, the antigen binding protein that binds OX40 and/or the antigen binding protein that binds PD-1 is a fully human monoclonal antibody.

The antigen binding protein that binds OX40 is an antibody with an IgG1 isotype or variant thereof. In one embodiment, the antigen binding protein that binds PD-1 is an antibody with an IgG1 isotype or variant thereof. The antigen binding protein that binds OX40 is an antibody with an IgG4 isotype or variant thereof. In one embodiment, the antigen binding protein that binds PD-1 is an antibody with an IgG4 isotype or variant thereof. In one aspect the antigen binding protein that binds OX40 is an agonist antibody. In one aspect the antigen binding protein that binds PD-1 is an antagonist antibody.

Suitably, the antigen binding protein that binds OX40 comprises: a heavy chain variable region CDR1 comprising an amino acid sequence with at least 90% 91%, 92%, 93%, 94%, 95%, 96%, 97%, 98%, 99% k or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

Suitably, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

Suitably, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

Suitably, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

Suitably, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23. Suitably, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 or 17. Suitably, the antigen binding protein that binds OX40 comprises a heavy chain variable region comprising the amino acid sequence set forth in SEQ ID NO:5 and a light chain variable region comprising the amino acid sequence set forth in SEQ ID NO:11.

Suitably, the antigen binding protein that binds OX40 comprises a heavy chain variable region comprising the amino acid sequence set forth in SEQ ID NO:17 and a light chain variable region comprising the amino acid sequence set forth in SEQ ID NO:23. Suitably, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23. Suitably, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In one embodiment, the antigen binding protein that binds PD-1 is pembrolizumab, or an antibody comprising 90%, 91%, 92%, 93%, 94%, 95%, 96%, 97%, 98%, 99% or 100% sequence identity thereto. In another embodiment, the antigen binding protein that binds PD-1 is nivolumab, or an antibody having 90%, 91%, 92%, 93%, 94%, 95%, 96%, 97%, 98%, 99% or 100% sequence identity thereto.

In one aspect, the mammal has increased survival when treated with a therapeutically effective amount of an antigen binding protein to OX40 and therapeutically effective amount of an antigen binding protein to PD-1 compared with a mammal who received the antigen binding protein to OX40 as a monotherapy or the antigen binding protein to PD-1 as a monotherapy. In one aspect, the methods further comprise administering at least one anti-neoplastic agent to the mammal in need thereof.

In one embodiment, pharmaceutical compositions are provided comprising a therapeutically effective amount of an antigen binding protein that binds OX40 and a therapeutically effective amount of an antigen binding protein that binds PD-1.

In one embodiment, the pharmaceutical compositions comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO:1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO:2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO:3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO:7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO:8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO:9; and pembrolizumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and pembrolizumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO:1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO:2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO:3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO:7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO:8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO:9; and nivolumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and nivolumab (heavy chain SEQ ID NO:98, light chain SEQ ID NO:99), or an antibody comprising 90%, 91%, 92%, 93%, 94%, 95%, 96%, 97%, 98%, 99% or 100% sequence identity thereto.

Also provided in the present invention are the use of a combination or pharmaceutical compositions of this invention in the manufacture of a medicament for the treatment of cancer. In some embodiments, the use futher includes radiotherapy. Also provided are the use of pharmaceutical compositions of the present invention for treating cancer. The present invention also provides combination kit comprising pharmaceutical compositions of the invention together with one or more pharmaceutically acceptable carriers.

In one embodiment methods are provided for reducing tumor size in a human having cancer comprising administering a therapeutically effective amount of an agonist antibody to human OX40 and a therapeutically effective amount of an antagonist antibody to human PD-1. In some embodiments, the use futher includes radiotherapy.

Radiotherapy

Radiotherapy is the use of high-energy radiation from x-rays, gamma rays, neutrons, protons, and other sources to kill cancer cells and shrink tumors. Radiotherapy may also be called irradiation and radiation therapy.

X-rays, gamma rays, and charged particles are examples of types of radiation used for cancer treatment.

The radiation may be delivered by a machine outside the body (external-beam radiation therapy (XRT)), or it may come from radioactive material placed in the body near cancer cells (internal radiation therapy, also called brachytherapy).

Systemic radiation therapy uses radioactive substances, such as radioactive iodine or a radiolabeled monoclonal antibody, that travel in the blood and/or to tissues thoughout the body to kill cancer cells.

Radiotherapy includes external-beam radiation therapy; internal radiation therapy (brachytherapy), and systemic radiation therapy. Types of external-beam radiation therapy include: Intensity-modulated radiation therapy (IMRT), Image-guided radiation therapy (IGRT), Tomotherapy, Stereotactic radiosurgery, Stereotactic body radiation therapy, Proton therapy, and other charged particle beams.

Radiation therapy is a primary therapy for patients with inoperable localized non-small cell lung carcinoma. However, there is a high rate of local failure, and while it increases median survival, the therapy is often not curative. Standard radiation fractionation provides a daily dose on the order of 1.8-2Gy, to a final dose of 60-70Gy. By contrast, Stereotactic Body Radiation Therapy (SBRT) is a relatively novel technique in radiation therapy of lung carcinomas, delivering the total dose in 5 or fewer treatments of radiation (hypofractionation). Response rates in clinical trials suggest SBRT could be an important therapeutic advance. This approach may have significant relevance to the endogenous immune response, since lymphocytes are sensitive to even low radiation doses and are cleared rapidly from the radiation field. Standard fractionated radiation treatment may limit the effectiveness of the immune system by constantly removing tumor antigen-specific T cells at the target site. Thus, although standard fractionation has been shown to generate endogenous anti-tumor immune responses, SBRT hypofractionation may be a more optimal partner for immunotherapy. In traditional external beam radiation therapy coupled with radiosensitizer administration, a beam of high energy X-rays, generated outside the patient by a linear accelerator, is delivered to a tumor. Most body tissue does not absorb or block X-rays, so they progress through the body, constantly releasing energy. When the cancer tumor is within the path of the X-ray, it receives some of that radiation; however, surrounding healthy tissue receives radiation as well. In order to limit the extent of collateral tissue damage, oncologists typically bombard the tumor area with the lowest level of effective radiation from many different points of entrance in an attempt to minimize damage to normal tissues. Even modem external beam radiation systems with improved real-time imaging of the patient anatomy will inevitably treat substantial normal tissue volumes when targeting the tumor.

Other energy sources, such as particle beams contain charged atomic particles. Particle beams have tremendous energy but also high mass and as such they slow down as they encounter body tissue. Particles can be controlled, for example, to release their energy at a specific point in the body. Particle beam therapy uses electrons, neutrons, heavy ions (such as protons, carbon ions and helium); and pi-mesons (also called pions).

Recent approaches to radiotherapy use high-dose radiation with precise focus on the cancerous area, limiting exposure of healthy cells to radiation. Stereotactic Body Radiation Therapy (“SBRT”), uses image-guided, focused high-dose external beam x-ray radiation to irradiate a tumor, often in a single fraction. To avoid the excessive toxicity which can occur to normal tissue, however, many tumors, even when targeted with SBRT, must be irradiated over two to five fractions, each fraction of lower dose than single fraction SBRT. The reduced dose per SBRT fraction may not be adequate to destroy the hypoxic component of the tumor.

Stereotactic radiosurgery (“SRS”), is a non-surgical procedure that delivers a single high-dose of precisely-targeted radiation typically targeted to the brain, head and neck using highly focused gamma-ray or x-ray beams that converge on the specific area or areas where the tumor resides, minimizing the amount of radiation to healthy tissue. Although stereotactic radiosurgery is often completed in a one-day session, physicians sometimes recommend multiple treatments, especially for tumors larger than one inch in diameter. The procedure is usually referred to as fractionated stereotactic radiosurgery when two to five treatments are given and as stereotactic radiotherapy when more than five treatments are given.

Intraoperative Radiation Therapy (“IORT”) is the delivery of radiation at the time of surgery using a focused high-dose radiation directed to the site of the cancerous cells. IORT is characterized by a concentrated beam of ionizing radiation to cancerous tumors while the patient is exposed during surgery, i.e., radiation is delivered within an open body cavity. IORT has an advantage of being able to temporarily displace healthy tissue from the path of the radiation beam so as to reduce the exposure of normal tissues to the radiation and contact the tumor site more directly. Single dose IORT in excess of 8-10 Gy, is effective at destroying tumor stem cells and its host-derived microvascular structure, thereby inhibiting DNA repair in the tumor, but hypoxic cells within the tumor may require doses in excess of 20-24 Gy, doses that could exceed normal tissue tolerance.

Radiotherapy of the invention may comprise a cumulative external irradiation of a patient in a dose of 1 to 100 Gy. A preferred range of the irradiation dose is 1 to 60 Gy. In certain embodiments, the dose of radiation therapy is less than 90 Gy, such as less than 80 Gy, such as less than 70 Gy, such as less than 60 Gy, such as less than 50 Gy, such as less than 40 Gy, such as less than 30 Gy, such as less than 20 Gy. In certain embodiments the dose or radiation therapy is between about 10 to 100 Gy, such as from about 20 to 80 Gy, such as about 30 to 70 Gy, such as about 40 to 60 Gy. In certain embodiments, the irradiation dose is selected from 5-25 Gy, such as from 10-20 Gy.

Radiation therapy may be stereotactic body radiotherapy, or SBRT. Stereotactic radiotherapy uses essentially the same approach as stereotactic radiosurgery to deliver radiation to the target tissue; however, stereotactic radiotherapy generally uses multiple small fractions of radiation as opposed to one large dose, but certain applications of SBRT may still be accomplished with a single fraction. Stereotactic body radiotherapy may be used to treat tumors in the brain, lung, liver, pancreas, prostate, spine, as well as other parts of the body.

Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy, etc.

As described herein, an anti-OX40 ABP (e.g., an agonist antibody, e.g., an agonist antibody described herein) is used in the treatment of a cancer, e.g., an anti-PD-1 resistant cancer, in combination with radiation therapy. In some embodiments, an anti-PD-1 ABP (e.g., an antagonist antibody, e.g., an antagonist antibody described herein) is included in the combination.

In some embodiments, the radiation therapy used in combination with an anti-OX40 ABP (e.g., an agonist antibody, e.g., an agonist antibody described herein) is SBRT. In some embodiments, an anti-PD-1 ABP (e.g., an antagonist antibody, e.g., an antagonist antibody described herein) is included in the combination.

In one embodiment, an anti-OX40 ABP (e.g., an agonist antibody, e.g., an agonist antibody described herein) is used in the treatment of a cancer, e.g., an anti-PD-1 resistant cancer, in combination with radiation therapy. Suitable examples of radiation therapy include external beam radiotherapy (EBRT or XRT) or teletherapy, brachytherapy or sealed source radiotherapy, or systemic radioisotope therapy or unsealed source radiotherapy. In some embodiments, an anti-PD-1 ABP (e.g., an antagonist antibody, e.g., an antagonist antibody described herein) is included in the combination.

Methods of Treatment

The combinations of the invention are believed to have utility in disorders wherein the engagement of OX40 (e.g., agonistic engagement, e.g., with an agonist antibody, e.g., an agonist antibody described herein) and/or radiotherapy, is beneficial, e.g., for the treatment of a cancer, e.g., an anti-PD-1 resistant cancer.

Treatment of a subject with a cancer (e.g., an anti-PD-1 resistant cancer) with an anti-OX40 antigen binding protein and radiotherapy may cause an abscopal effect. As used herein, “abscopal effect” refers to a phenomenon in the treatment of metastatic cancer where localized treatment of a tumor causes not only a shrinking of the treated tumor, but also a shrinking of tumors outside the scope of the localized treatment.

Treatment of a subject with a cancer (e.g., an anti-PD-1 resistant cancer) with an anti-OX40 antigen binding protein and radiotherapy may sensitize the anti-PD-1 resistant cancer to anti-PD-1 therapy, e.g., the cancer will respond to anti-PD-1 therapy after treatment with an anti-OX40 antigen binding protein and radiotherapy, and/or the cancer will respond to anti-PD-1 therapy administered during treatment with an anti-OX40 antigen binding protein and radiotherapy.

The present invention thus also provides a combination of the invention (e.g., an anti-OX40 ABP and radiotherapy, optionally with an anti-PD-1 ABP), for use in therapy, particularly in the treatment of disorders wherein the engagement of OX40 (e.g., agonistic engagement, e.g., with an agonist antibody, e.g., an agonist antibody described herein) and/or radiotherapy, is beneficial, particularly cancer, e.g., for the treatment of an anti-PD-1 resistant cancer.

A further aspect of the invention provides a method of treatment of a disorder (e.g., for the treatment of a cancer, e.g., an anti-PD-1 resistant cancer) wherein engagement of OX40 (e.g., agonistic engagement, e.g., with an agonist antibody, e.g., an agonist antibody described herein) and/or radiotherapy, is beneficial, comprising administering a combination of the invention (e.g., an anti-OX40 ABP and radiotherapy, optionally with an anti-PD-1 ABP).

A further aspect of the present invention provides the use of a combination of the invention (e.g., an anti-OX40 ABP and radiotherapy, optionally with an anti-PD-1 ABP) in the manufacture of a medicament for the treatment of a disorder engagement of OX40 (e.g., agonistic engagement, e.g., with an agonist antibody, e.g., an agonist antibody described herein) and/or radiotherapy, is beneficial, e.g., for the treatment of a cancer, e.g., an anti-PD-1 resistant cancer.

The combinations of the invention (e.g., an anti-OX40 ABP and radiotherapy, optionally with an anti-PD-1 ABP) are believed to have utility in disorders wherein the engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein), in combination with radiotherapy, is beneficial, e.g., for the treatment of a cancer, e.g., an anti-PD-1 resistant cancer.

The present invention thus also provides a combination of the invention (e.g., an anti-OX40 ABP and radiotherapy, optionally with an anti-PD-1 ABP), for use in therapy, particularly in the treatment of disorders wherein the engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein), in combination with radiotherapy, is beneficial, particularly a cancer, e.g., for the treatment of an anti-PD-1 resistant cancer.

A further aspect of the invention provides a method of treatment of a disorder (e.g., for the treatment of a cancer, e.g., an anti-PD-1 resistant cancer) wherein engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein), in combination with radiotherapy, is beneficial, comprising administering a combination of the invention (e.g., an anti-OX40 ABP and radiotherapy, optionally with an anti-PD-1 ABP).

A further aspect of the present invention provides the use of a combination of the invention (e.g., an anti-OX40 ABP and radiotherapy, optionally with an anti-PD-1 ABP) in the manufacture of a medicament for the treatment of a disorder, wherein engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein), in combination with radiotherapy, is beneficial, e.g., for the treatment of a cancer, e.g., an anti-PD-1 resistant cancer.

The combinations of the invention (e.g., an anti-OX40 ABP and radiotherapy, optionally with an anti-PD-1 ABP) are believed to have utility in disorders wherein the engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein) and/or radiotherapy, is beneficial, e.g., for the treatment of a cancer, e.g., an anti-PD-1 resistant cancer.

The present invention thus also provides a combination of the invention (e.g., an anti-OX40 ABP and radiotherapy, optionally with an anti-PD-1 ABP), for use in therapy, particularly in the treatment of disorders wherein the engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein) and/or radiotherapy, is beneficial, particularly a cancer, e.g., for the treatment of an anti-PD-1 resistant cancer.

A further aspect of the invention provides a method of treatment of a disorder (e.g., for the treatment of a cancer, e.g., an anti-PD-1 resistant cancer) wherein engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein) and/or radiotherapy, is beneficial, comprising administering a combination of the invention (e.g., an anti-OX40 ABP and radiotherapy, optionally with an anti-PD-1 ABP) to a subject in need thereof.

A further aspect of the present invention provides the use of a combination of the invention (e.g., an anti-OX40 ABP and radiotherapy, optionally with an anti-PD-1 ABP) in the manufacture of a medicament for the treatment of a disorder wherein engagement of OX40 (e.g., agonistic engagement, e.g., with an agonist antibody, e.g., an agonist antibody described herein) and/or PD-1 (e.g., antagonistic engagement, e.g., with an antagonist antibody, e.g., an antagonist antibody described herein) and/or radiotherapy, is beneficial, e.g., for the treatment of a cancer, e.g., an anti-PD-1 resistant cancer.

Examples of cancers, e.g., that may be or may become anti-PD-1 resistant, that are suitable for treatment with a combination of the invention include, but are not limited to, both primary and metastatic forms of head and neck, breast, lung, colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Additionally, examples of a cancer, e.g., that may be or may become anti-PD-1 resistant, to be treated include Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer, e.g., that may be or may become anti-PD-1 resistant, selected from the group consisting of: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer, e.g., that may be or may become anti-PD-1 resistant, selected from the group consisting of: ovarian, breast, pancreatic and prostate cancer.

Suitably, the present invention relates to a method for treating or lessening the severity of non-small cell lung carcinoma (NSCLC), small cell lung cancer (SCLC), bladder cancer or metastatic hormone-refractory prostate cancer, e.g., in each case, that may be or may become anti-PD-1 resistant.

Suitably, the present invention relates to a method for treating or lessening the severity of melanoma, e.g., metastatic melanoma that may be or may become anti-PD-1 resistant.

Suitably, the present invention relates to a method for treating or lessening the severity of lung cancer, e.g., lung cancer that may be or may become anti-PD-1 resistant.

Suitably the present invention relates to a method for treating or lessening the severity of pre-cancerous syndromes in a mammal, including a human, wherein the pre-cancerous syndrome is selected from the group consisting of: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps, severe hepatitis, and cirrhosis, in each case, that may be or may become anti-PD-1 resistant.

The combination of the invention may be used alone or in combination with one or more other therapeutic agents. The invention thus provides in a further aspect a further combination comprising a combination of the invention with a further therapeutic agent or agents, compositions and medicaments comprising the combination and use of the further combination, compositions and medicaments in therapy, in particular in the treatment of diseases susceptible engagement of OX40 (e.g., agonism of OX40), and radiotherapy and/or engagement of PD-1 (e.g., antagonism of PD-1).

In the embodiment, the combination of the invention may be employed with other therapeutic methods of cancer treatment, e.g., with a further anti-cancer therapy. In particular, wherein the combnation with another the anti-cancer therapy is a combination with an anti-neoplastic therapy (e.g., an anti-neoplastic agent), combination therapy with other chemotherapeutic, hormonal, antibody agents as well as surgical and/or radiation treatments other than those mentioned above are envisaged. Combination therapies according to the present invention thus include the administration of an anti-OX40 ABP of a combination, or method or use thereof, of the invention and radiotherapy and/or an anti-PD-1 ABP of a combination, or method or use thereof, of the invention as well as optional use of other therapeutic agents including other anti-neoplastic agents. The term “combination” refers to the use of the two or more therapies to treat the same patient (subject) for a reason(s) related to the same indication (e.g., the therapies of the combination are used to treat the same indication or an indication and side effect(s) or symptom(s) related thereto), wherein the use or actions of the therapies overlap in time. The therapies can be administered at the same time (e.g., as a single formulation that is administered to a patient or as two separate formulations or treatments administered concurrently) or sequentially in any order. Sequential administrations are administrations that are given at different times. The time between administration of the one therapy and another therapy can be minutes, hours, days, or weeks. For example, the time between administration of the one therapy and another therapy is 12, 24, 36, 48, 60, 72, 84, or 96 hours. For example, the time between administration of an anti-OX40 ABP and radiotherapy is 12, 24, 36, 48, 60, 72, 84, or 96 hours. For example, an anti-OX40 ABP can be administered 12, 24, 36, 48, 60, 72, 84, or 96 hours after radiotherapy.

In one embodiment, the pharmaceutical combination includes an anti-OX40 ABP, suitably an agonist anti-OX40 ABP, and optionally at least one additional anti-neoplastic agent for use (simultaneously or sequentially) with radiotherapy. In one embodiment, the pharmaceutical combination includes an anti-OX40 ABP, suitably an agonist anti-OX40 ABP and an anti-PD-1 ABP, suitably an antagonist anti-PD-1 ABP, and optionally at least one additional anti-neoplastic agent for use (simultaneously or sequentially) with radiotherapy. In one embodiment, the pharmaceutical combination includes an anti-OX40 ABP, suitably an agonist anti-OX40 ABP and radiotherapy, and optionally at least one additional anti-neoplastic agent.

In one embodiment, the further anti-cancer therapy is surgical.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the GilM phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273,1989) and for the treatment of breast cancer (Holmes et al., J. Nat. Cancer Inst., 83:1797,1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q. v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R-(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes: Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinurn, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)aminc]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma.

Daunorubicin, (8S-cis+8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents: Anti-angiogenic agents including non-receptorMEKngiogenesis inhibitors may alo be useful. Anti-angiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin);

Immunotherapeutic agents: Agents used in immunotherapeutic regimens may also be useful in combination with the compounds of formula (I). Immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Proapoptotoc agents: Agents used in proapoptotic regimens (e.g., bcl-2 antisense oligonucleotides) may also be used in the combination of the present invention.

Cell cycle signalling inhibitors: Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the combination of the present invention comprises an anti-OX40 ABP optinally with a PD-1 modulator (e.g., anti-PD-1 ABP) and/or radiotherapy and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-OX40 ABP optionally with a PD-1 modulator (e.g., anti-PD-1 ABP) and/or radiotherapy and at least one anti-neoplastic agent which is an anti-microtubule agent selected from diterpenoids and vinca alkaloids.

In a further embodiment, the at least one anti-neoplastic agent agent is a diterpenoid.

In a further embodiment, the at least one anti-neoplastic agent is a vinca alkaloid. In one embodiment, the combination of the present invention comprises an anti-OX40 ABP optionally with a PD-1 modulator (e.g., anti-PD-1 ABP) and/or radiotherapy and at least one anti-neoplastic agent, which is a platinum coordination complex.

In a further embodiment, the at least one anti-neoplastic agent is paclitaxel, carboplatin, or vinorelbine.

In a further embodiment, the at least one anti-neoplastic agent is carboplatin.

In a further embodiment, the at least one anti-neoplastic agent is vinorelbine.

In a further embodiment, the at least one anti-neoplastic agent is paclitaxel.

In one embodiment, the combination of the present invention comprises an anti-OX40 ABP optionally with a PD-1 modulator (e.g., anti-PD-1 ABP) and/or radiotherapy and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

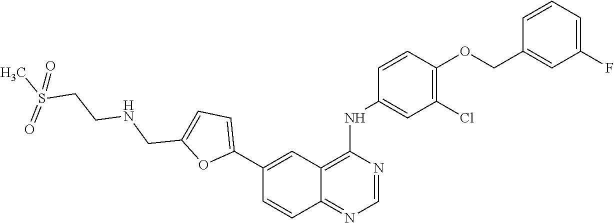 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of formula I or a salt or solvate thereof and at least one anti-neoplastic agent which is a cell cycle signaling inhibitor.

In further embodiment, cell cycle signaling inhibitor is an inhibitor of CDK2, CDK4 or CDK6.

In one embodiment the mammal in the methods and uses of the present invention is a human.

As indicated, therapeutically effective amounts of the combinations of the invention (an anti-OX40 ABP, optionally with a PD-1 modulator (e.g., anti-PD-1 ABP) and/or radiotherapy), are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

Anti-PD-1 Resistance

Immunotherapies targeting PD1/PDL1 (such as an anti-PD-1 antigen binding protein) have shown good rates of durable clinical responses in cancer patients, e.g., with melanoma and lung cancer. However, there is a substantial portion of subjects (e.g., patients) who do not respond to these therapies, e.g., a large number of patients present with or develop resistance to them. See, e.g., O'Donnell et al., Genome Medicine 8:111 (2016) and Wang et al., Cancer Res 77:1-12 (2017). For example, a portion of subjects with a cancer that was responsive (e.g., the cancer was decreasing in size, severity and/or metastases) to anti-PD1 treatment stops responding to the treatment, e.g., the cancer increases in size, severity and/or metastases while the anti-PD1 treatment is being administered to the subject.

In one study, approximately 25% of patients with melanoma who had had an objective response to PD-1 blockade therapy had disease progression at a median follow-up of 21 months (see, e.g., Zaretsky et al., N Engl J Med 375:819-829 (2016)).

Subjects and cancers that present with or develop resistance to immunotherapies targeting PD1/PDL1 are considered to be anti-PD-1 resistant.

The following example is intended for illustration only and are not intended to limit the scope of the invention in any way.

### EXAMPLES

### Summary

129sv/ev mice bearing subcutaneously implanted anti-PD-1 resistant 344SQ mouse lung adenocarcinoma cells on both flanks were treated by intratumoral injection of the primary tumor with the murine monoclonal antibody (mAb) against OX40 (OX86, rat IgG1mAb) alone or following radiation to the same tumor. The aim of this work was to determine if treatment could overcome anti-PD-1 resistance and what effect these treatments might have on abscopal tumor control. Treatment with either five 200 pg doses of OX86 alone or in combination with 12Gy*3 of radiation resulted in a significantly lower mean volume of both the primary and secondary tumors versus control IgG1. The combination of the OX40 agonist mAb with radiotherapy also increased survival. Furthermore, treatment with adjuvant radiation therapy with anti-OX40 mAb showed increased tumor control compared to anti-OX40 alone. The combination of OX40 mAb and radiotherapy was found to be advantageous for abscopal effects with reduction in lung metastasis. Radiation alone was shown to significantly increase the percentage of OX40 positive CD4 T helper cells in both spleens and tumors of treated mice as well as increase T cell activating CD103+ dendritic cells in the spleen.

### 1. INTRODUCTION

OX40 is a co-stimulatory molecule expressed primarily on activated effector T cells (activated CD4+ T cells and CD8+ T cells) and naive regulatory T cells. OX40 ligand (OX40L; CD252) is expressed on activated professional antigen presenting cells such as dendritic cells (DCs), macrophages, and B cells (3, 4). Ligation of OX40 on CD4+ T cells activates the NF-κB pathway and up-regulates anti-apoptotic molecules of the Bcl-2 family which play a role in T cell expansion, activation, memory, and cytokine production (5, 7). In this study, adjuvant radiation therapy was combined with an OX40 agonist mAb cancer immunotherapy agent. The hypothesis for this study is that radiation therapy induces a local inflammatory response that could enhance the infiltration of tumor-specific T cells and simultaneously induce OX40 expression in the tumor microenvironment. These events are known to markedly induce anti-tumor immunity. The concept of radiation-induced OX40 expression in an anti-PD1 resistant microenvironment might expand the application of an OX40 agonist to include combination with radiation therapy.

The purpose of this study was to determine whether treatment with an anti-OX40 mAb administered intratumorally alone or in combination with radiation could overcome anti-PD-1 resistance in a preclinical lung cancer model and to examine both abscopal effects on untreated tumors and pharmacodynamic changes on immune cells in various compartments.

### 2. MATERIALS AND METHODS

All procedures on animals were reviewed and approved by the MD Anderson Animal Care and Use Committee prior to initiation of the studies.

2.1. Experimental Preparation(s)

2.1.1. Preparation of Anti-PD-1 Resistant 3445Q Mouse Lung Cancer Cells

The 344SQ parental cell line (344SQ_P) is a metastatic mouse lung cancer cell line derived from a spontaneous subcutaneous metastatic lesion in p53^{R172HΔg/+}K-ras^{LA1/+} mice (6). This anti-PD-1-resistant cell line, 344SQ-R, was generated as described previously. Cell lines were cultured in complete media [CM; RPMI1640 supplemented with 100 units/mL penicillin, 100 pg/mL streptomycin, 10 mmol/L L-glutamine, and 10% heat-inactivated fetal bovine serum (all reagents from Sigma Aldrich)] in a humidified incubator at 37° C. and 5% CO₂.

2.1.2. 129Sv/Ev Subcutaneous Injection

The mice used in this study were female 129Sv/Ev purchased from Taconic. Mice were injected with tumors at 8-12 weeks of age, and each experiment used mice of the same age. All mice were housed at the Experimental Radiation Oncology (ERO) mouse colony facility at The University of Texas, MD Anderson Cancer Center (MDACC) Animal Care and were cared for accordingly. Whole procedures were revised and accepted by MDACC Animal Care.

Tumors were established by subcutaneous injection using 26 gauge needles on the right flank (0.5×10⁶ cells/100 μl PBS per mouse) on day 0, and for assessment of abscopal effect, 0.1×10⁶ tumor cells SC into the left flanks on day 4. Five days before treatment, each mouse was tagged on the right ear. The mice were randomized, divided into separate cohorts and subjected to different treatments.

2.1.3. Antibody Preparation

Therapeutic anti-OX40 antibodies (Clone OX86; Catalog#13E0031) and the control rat IgG1 antibodies (Clone2A3; Catalog#:BE0088) were diluted to 2 mg/mL in sterile PBS without Ca and Mg. Treatment solutions were prepared aseptically immediately prior to administration.

2.2. Materials: Drugs and Reagents

| | | Catalogue/batch |
|---|---|---|
| Reagent | Supplier | number |
| Anti-PD-1 resistant 344SQ | Generated in Dr. Welsh | Cryopreserved |
| murine lung cancer cells | lab | |
| RPMI1640 | GE Healthcare Life | SH30027.01/AZA193908 |
| | Sciences | |
| Fetal Bovine Serum | Sigma | F4135-500 ml/15A358 |
| Penicillin-Streptomycin | Corning | 30-002-CI/30002242 |
| Solution, 100X | | |
| Trypsin-EDTA solution | Sigma Aldrich | F4049-100 ml/SLBM51201 |
| Sterile 1xPBS without Ca or | In-house | MFB004/batch # not |
| Mg | | provided |
| Rat anti-mouse OX40 | BioXcell | BE0031 5534/1214 |
| (OX86) | | |
| rat IgG1 mAb isotype | BioXcell | BE0088 5339/1014 |
| control 7.3 | | |
| Anti-CD8 | Biolegend | #100734 |
| Anti-CD45 | Biolegend | #103126 |
| Anti-CD11b | Biolegend | #101226 |
| Anti-CD11c | Biolegend | #117310 |
| Anti-F4/80 | Biolegend | #123108 |
| Anti-Ly6C | Biolegend | #128018 |
| Anti-Ly6G | Biolegend | #127616 |
| anti-CD103 | Biolegend | #121426 |
| anti-CD3 | Biolegend | #100353 |
| anti-CD4 | Biolegend | #100406 |
| anti-OX40 | Biolegend | #119414 |
| anti-OX40L | Biolegend | #108805 |

2.3. Experimental Protocol(s)

The purpose of this study was to determine whether treatment with an anti-murine OX40 mAb (OX86) administered intratumorally alone or in combination with radiation could overcome anti-PD-1 resistance in a preclinical lung cancer model and to examine both abscopal effects on untreated tumors and pharmacodynamic changes on immune cells in various compartments.

Tumor size was assessed every other day and recorded in mm³. The tumor was measured with calipers, and tumor volume (V) was calculated by measuring length (L) and width (W) as: V=W²×L/2. Mice were sacrificed when tumors became ulcerated or reached a maximum size of 1500 mm³.

An overview of the experimental protocol for the efficacy portion of this work is shown in FIG. 13. Briefly, tumor-bearing mice were randomized into 4 cohorts. The primary tumor was injected intratumorally with anti-OX40 antibody or rat IgG isotype control antibody either alone or following three treatments of 12 Gy radiation. Mice were monitored for 60 days to investigate primary and secondary tumor growth and survival rate.
Cohort 1: IgG Control—5 doses of 200 ug, 2× per week (n=10 mice)
Cohort 2: anti-OX86—5 doses of 200 ug, 2× per week (n=10 mice)
Cohort 3: 3*12Gy radiation followed by IgG Control—5 doses of 200 ug, 2× per week (n=7 mice)
Cohort 4: 3*12Gy radiation followed by OX86—5 doses of 200 ug, 2× per week (n=11 mice)

The pharmacodynamics of these treatments were studied by harvesting spleens and tumors to be characterized by flow cytometry. Additionally, lungs were harvested for quantitating metastases. Mice were implanted with 344SQ anti-PD1 resistant tumors and treated with radiation and OX86 in the same manner as described in the efficacy study. Animals were sacrificed at day 32, 2 days post the final OX86 or isotype treatment. Tumors were harvested from 3 out of 6 mice.

To obtain single-cell suspensions, tumor tissues were digested with 1 mg/ml collagenase IV (Sigma-Aldrich) and for 45 minutes at 37° C. Spleens were collected, processed into a single cell suspension, and filtered with 70 pmpm filters. Suspensions were treated with ACK lysis buffer. Before all staining, cells were Fc blocked with anti-CD16/CD32. Cells were stained with antibodies against CD4, CD8, CD45, CD11b, CD11c, F4/80, Ly6C, Ly6G, OX40 and OX40L and acquired using an LSR II flow cytometer. Data were analyzed using FlowJo software.

Lungs were harvested from mice from each cohort for quantification of lung metastases. Lungs were collected at the end of the experiment, fixed in Bouin's solution (Sigma) for 3 days and lung metastases were counted manually.
Cohort 5: IgG Control—5 doses of 200 ug, 2× per week (n=6 mice)
Cohort 6: anti-OX86—5 doses of 200 ug, 2× per week (n=6 mice)
Cohort 7: 3*12Gy radiation followed by IgG Control—5 doses of 200 ug, 2× per week (n=6 mice)
Cohort 8: 3*12Gy radiation followed by OX86—5 doses of 200 ug, 2× per week (n=6 mice)

A radiation only study was also conducted to assess the effect of radiation on splenocytes and tumor infiltrating lymphocytes at various timepoints post-treatment. Mice were implanted and randomized and treated with radiation as in previously described studies. The first group of mice were harvested 48 hours following the final dose of radiation, and splenocytes were immunophenotyped. The second group was sacrificed 7 days following the final dose and both splenocytes and tumor infiltrating lymphocytes were immunophenotyped by flow. Splenocytes and tumors were processed as decribed above.
Cohort 9: No treatment, harvested at 48 hrs post Cohort 10 final radiation dose (n=3)
Cohort 10: 3*12Gy radiation, harvested at 48 hrs post final radiation dose (n=3)
Cohort 11: No treatment, harvested at 7 days post Cohort 12 final radiation dose (n=3)
Cohort 12: 3*12Gy radiation, harvested at 7 days post final radiation dose (n=3)

2.4. Data Analysis

Results for the efficacy study portion of the work were expressed as mean 6 SEM. The tumor volumes between each individual treatment group at different measurement days (day 9 to 60) were compared in Graph Pad Prism 6 using multiple t tests (desired false discovery rate (FDR)=1% and corrected for multiple comparison using the Holm-Sidak method and alpha=0.05). Data points with p-values 0.05 are declared to be statistically significant. Survival rates were analyzed using the Kaplan-Meier method and evaluated with the log-rank test with Bonferroni correction.

The pharmcodynamic portion of the work as well as the quantification of the lung metastais was statistically analyzed using One Way ANOVA with Holm-Sidak correction with the exception of the dendritic cell analysis which used a t test with Welsh correction. If statistics tables are not shown, none of the changes were found to be significant.

### 3. RESULTS

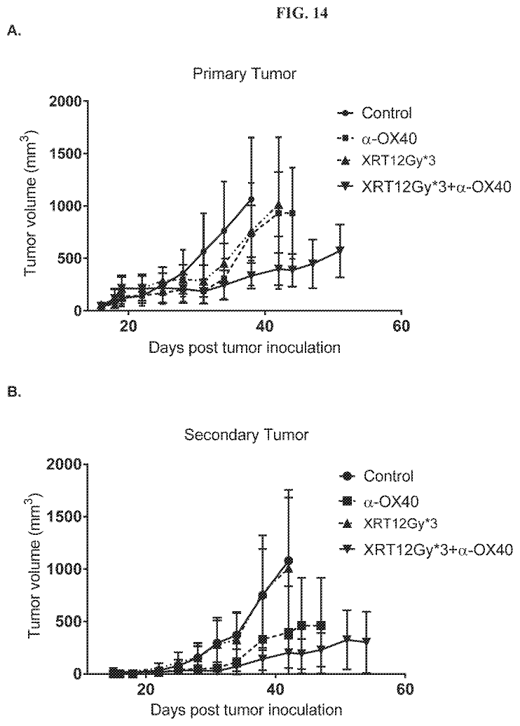

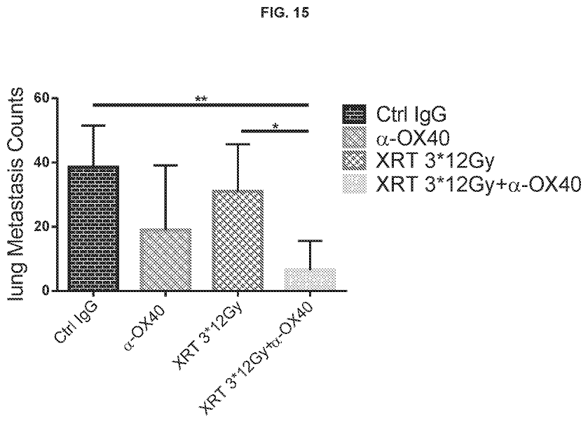

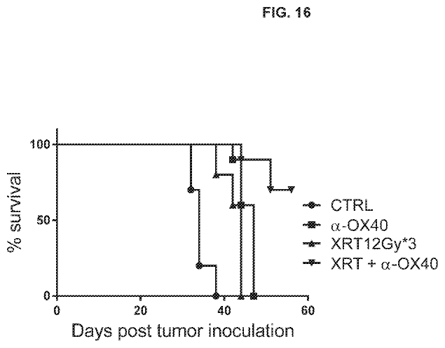

Intratumoral injections of OX40 agonist antibody (OX86), either alone or in combination with radiation, demonstrated in vivo activity against 344SQ PD-1 resistant tumors. These data show that the combination of OX86 with radiotherapy resulted in significantly decreased primary and secondary tumor volumes compared to the isotype control or either OX86 or radiation treatment alone (FIGS. 14A and B, Tables 1, 2). This combination also led to increased survival rates (FIG. 16). Quantification of lung metastases showed that the combination of OX86 and radiation significantly decreased metastases compared to isotype control as well as radiation alone (FIG. 15, Table 5).

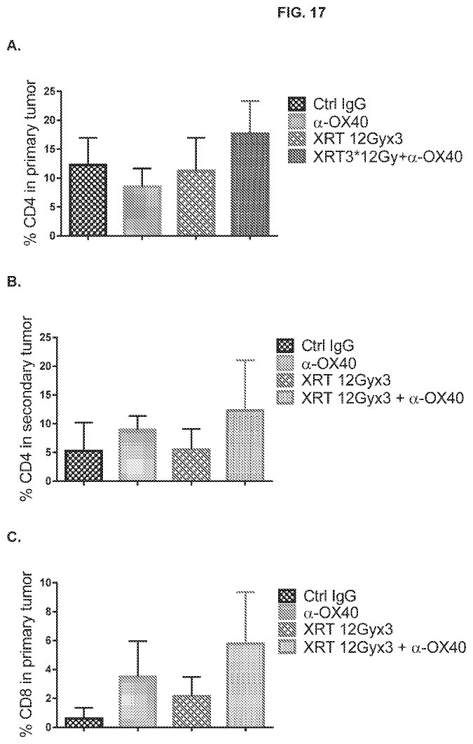

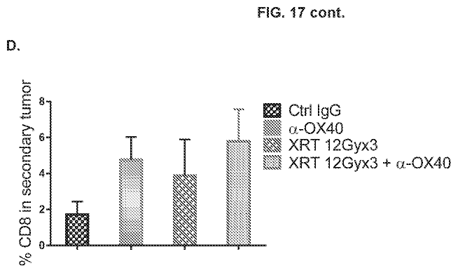

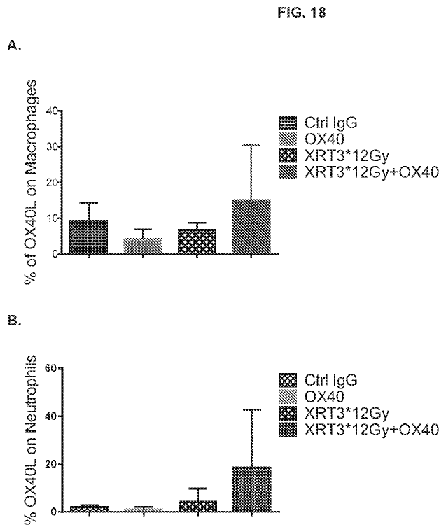

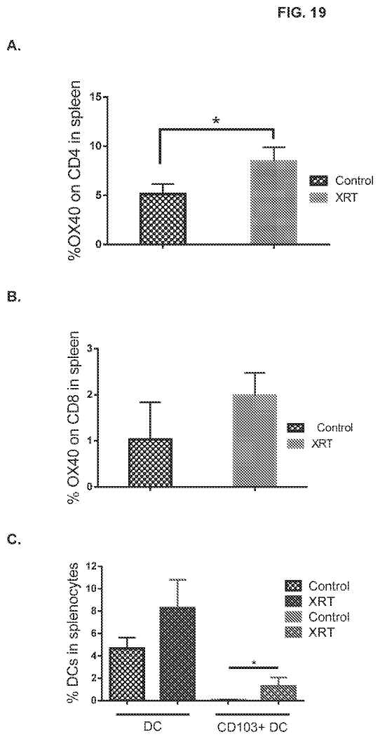

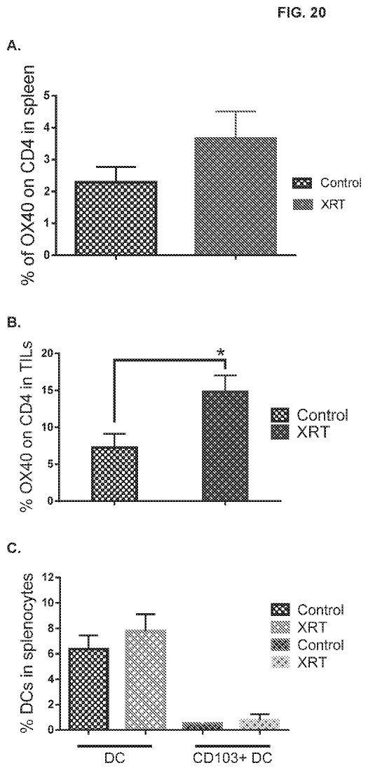

At 48 hours and 7 days post treatment with the combination of radiation and OX86, trends were observed toward increased infiltration of CD4 T cells in both the primary and secondary tumors and of CD8 T cells in both tumors of mice treated with OX86 alone or in combination with radiation (FIGS. 17A-D, Tables 6-9). Additionally, there were trends toward increased expression of OX40 ligand on both macrophages and neutrophils from primary tumors 48 hours post combination treatment (FIGS. 18A and B, Tables 16, 17). A significant increase in the percentage of OX40 positive CD4 cells in the spleens of radiation treated mice was observed 48 hours following 12*3 Gy radiation alone as well as trends toward increased expression in CD8 T cells in spleens at the same timepoint (FIGS. 19A and B, Tables 11, 12). At 7 days post treatment with radiation, OX40 positive CD4 T cells were significantly increased in tumors and trended higher in spleens (FIGS. 20A and B, Tables 14, 15). The overall percentages of dendritic cells trended higher in spleens at both 48 hours and 7 days post treatment with a significant increase in the CD103+ dendritic cells 48 hours post radiation (FIG. 19C, FIG. 20C; Tables 10, 13).

Additional data are provided in Tables 3 and 4.

### 4. DISCUSSION

These results suggest that the combination of anti-OX40 agonist antibody and radiation could potentially be utilized to overcome anti-PD-1 resistance and increase survival. Radiation alone was shown to significantly increase the percentage of OX40 positive CD4 T helper cells in both spleens and tumors of treated mice. Radiation also significantly increased T cell activating CD103+ DCs in the spleen. Trends toward increased CD4 and CD8 T cell infiltration into tumors was observed in mice treated with the combination of radiation and anti-OX40 antibody.

### 5. REFERENCES

1. Annual Review of Immunology. 2010; 28:57-78. Croft M. Control of immunity by the TNFR-related molecule OX40 (CD134).
2. Yokouchi H, Yamazaki K, Chamoto K, Kikuchi E, Shinagawa N, Oizumi S, et al. Anti-OX40 monoclonal antibody therapy in combination with radiotherapy results in therapeutic antitumor immunity to murine lung cancer. Cancer Science. 2008; 99(2):361-7.
3. Pan P Y, Zang Y, Weber K, Meseck M L, Chen S H. OX40 ligation enhances primary and memory cytotoxic T lymphocyte responses in an immunotherapy for hepatic colon metastases. Molecular therapy: The journal of the American Society of Gene Therapy. 2002; 6(4):528-36.
4. Linch S N, McNamara M J, Redmond W L. OX40 Agonists and Combination Immunotherapy: Putting the Pedal to the Metal. Frontiers in Oncology. 2015; 5:34.
5. Song J, So T, Croft M. Activation of NF-kappaB1 by OX40 contributes to antigen-driven T cell expansion and survival. Journal of Immunology. 2008; 180(11):7240-8.
6. Gibbons D L, Lin W, Creighton C J, Rizvi Z H, Gregory P A, Goodall P A, Goodall G J et al. Contextual extracellular cues promote tumor cell EMT and metastasis by regulating miR-200 family expression. Genes & Development. 2009; 23(18):2140-2151.
7. Jensen S M, Maston L D, Gough M J, Ruby C E, Redmond W L, Crittenden M et al. Signaling through OX40 enhances antitumor immunity. Seminars in Oncology. 2010; 37(5):524-532.

### 6. ABBREVIATIONS

| | |
|---|---|
| PD-1 | Programmed death 1 |
| TIL | tumor-infiltrating lymphocyte |
| MDACC | MD Anderson Cancer Center |
| ERO | Experimental radiation oncology |
| FBS | Fetal bovine serum |
| mAb | Monoclonal antibody |
| PBS | Phosphate buffered saline |
| FDR | False discovery rate |
| IACUC | Institutional Animal Care and Use |
| | Committee |
| ID | Identification |

### 7. TABLES

TABLE 1
Tumor Growth in primary tumor
a) Tumor Growth in primary tumor (Control IgG Group)

| Days post | | Control group (Mice#1-10) | | | | | | | | | | |
|---|---|---|---|---|---|---|---|---|---|---|---|---|
| tumor inj. | ID# | 1686 | 1670 | 1786 | 1643 | 1518 | 1517 | 6683 | 1644 | 1520 | 1557 | Mean |
| 16 | | 24.5 | 45.6 | 9.4 | 21.4 | 35.4 | 48.6 | 35.4 | 6.3 | 5.0 | 56.3 | 28.8 |
| 18 | | 172.8 | 147.0 | 11.8 | 73.9 | 52.5 | 108.0 | 139.7 | 18.4 | 72.9 | 137.3 | 93.4 |
| 19 | | 189.4 | 115.3 | 18.4 | 45.6 | 68.1 | 202.6 | 201.1 | 18.4 | 128.9 | 199.7 | 118.7 |
| 22 | | 236.7 | 133.1 | 18.4 | 45.6 | 108.0 | 303.5 | 176.0 | 40.5 | 122.5 | 210.9 | 139.5 |
| 25 | | 245.7 | 307.1 | 30.4 | 60.5 | 202.5 | 288.0 | 406.1 | 72.6 | 289.0 | 578.8 | 248.1 |
| 28 | | 460.0 | 475.7 | 105.6 | 98.0 | 196.9 | 406.1 | 557.0 | 75.7 | 479.6 | 726.0 | 358.1 |
| 31 | | 622.9 | 600.0 | 192.0 | 364.5 | 428.7 | 496.1 | 793.5 | 56.3 | 726.0 | 1372.0 | 565.2 |
| 34 | | 727.4 | 1372.0 | 307.8 | 252.9 | 528.2 | 665.5 | 1372.0 | 166.5 | 847.0 | 1372.0 | 761.1 |

b) Tumor Growth in primary tumor (OX86 Group)

| Days post | OX86 (Mice#1-10) | | | | | | | | | | | |
|---|---|---|---|---|---|---|---|---|---|---|---|---|
| tumor inj. | ID# | 1668 | 1577 | 1658 | 1776 | 1521 | 1576 | 1511 | 1649 | 1667 | 1789 | Mean |
| 16 | | 28.0 | 32.0 | 15.8 | 15.8 | 15.8 | 73.1 | 40.5 | 24.5 | 15.8 | 24.5 | 28.6 |
| 18 | | 109.9 | 68.1 | 24.5 | 75.6 | 67.6 | 81.2 | 156.8 | 24.5 | 15.8 | 108.0 | 73.2 |
| 19 | | 116.2 | 95.1 | 48.6 | 158.8 | 110.3 | 235.3 | 252.9 | 65.6 | 83.2 | 196.9 | 136.3 |
| 22 | | 196.9 | 105.6 | 75.7 | 196.9 | 134.8 | 224.0 | 264.9 | 78.2 | 95.1 | 75.0 | 144.7 |
| 25 | | 201.6 | 147.0 | 68.1 | 283.8 | 196.9 | 389.2 | 70.7 | 90.1 | 127.4 | 108.0 | 168.3 |
| 28 | | 270.9 | 147.0 | 68.1 | 321.7 | 256.0 | 389.2 | 50.0 | 81.0 | 191.3 | 240.0 | 201.5 |
| 31 | | 176.8 | 153.6 | 40.0 | 364.5 | 210.9 | 352.4 | 70.3 | 50.0 | 216.4 | 240.0 | 187.5 |
| 34 | | 456.2 | 258.8 | 74.4 | 726.0 | 258.3 | 456.2 | 159.3 | 134.8 | 224.0 | 299.9 | 304.8 |
| 38 | | 500.0 | 695.8 | 484.0 | 1687.5 | 406.1 | 1575.0 | 544.5 | 370.0 | 340.6 | 695.8 | 729.9 |
| 42 | | 864.0 | 1171.9 | 544.5 | 2754.0 | 760.4 | | 628.2 | 383.6 | 445.5 | 828.0 | 931.1 |

c) Tumor Growth in primary tumor (Radiation Group)

| | |
|---|---|
| Days Post | Radiation |

| tumor Inj. | ID# | 1693 | 1790 | 1503 | 1785 | 1504 | 1523 | 1664 | Mean |
|---|---|---|---|---|---|---|---|---|---|
| 16 | | 28.0 | 15.8 | 17.1 | 45.6 | 109.9 | 75.6 | 105.6 | 56.8 |
| 18 | | 46.6 | 15.8 | 17.1 | 159.3 | 224.0 | 240.0 | 116.2 | 117.0 |
| 19 | | 154.7 | 50.0 | 127.4 | 252.9 | 240.0 | 383.6 | 270.9 | 211.4 |
| 22 | | 146.3 | 43.8 | 108.0 | 289.0 | 240.0 | 329.3 | 344.4 | 214.4 |
| 25 | | 196.9 | 68.4 | 283.5 | 428.7 | 256.0 | 329.3 | 428.7 | 284.5 |
| 28 | | 181.3 | 72.0 | 281.8 | 428.7 | 275.2 | 428.7 | 428.7 | 299.5 |
| 31 | | 240.0 | 65.0 | 296.2 | 171.0 | 451.3 | 500.0 | 262.4 | 283.7 |
| 34 | | 489.8 | 109.9 | 360.5 | 734.2 | 451.3 | 550.0 | 460.3 | 450.8 |
| 38 | | 756.3 | 272.0 | 665.5 | 792.0 | 1044.0 | 859.4 | 925.8 | 759.3 |

d) Tumor Growth in primary tumor (Radiation + OX86 Group)

| Days post | Radiation + OX86 | | | | | | | | | | | | |
|---|---|---|---|---|---|---|---|---|---|---|---|---|---|
| tumor Inj. | ID# | 1525 | 1694 | 1542 | 1501 | 1671 | 1653 | 1791 | 1673 | 1792 | 1641 | 1505 | Mean |
| 16 | | 75.6 | 24.5 | 60.5 | 18.4 | 134.8 | 40.5 | 40.5 | 0.0 | 18.0 | 60.5 | 18.0 | 44.7 |
| 18 | | 171.5 | 81.7 | 144.5 | 129.6 | 262.4 | 108.9 | 60.0 | 0.0 | 43.8 | 0.0 | 256.0 | 114.4 |
| 21 | | 303.2 | 196.9 | 315.9 | 122.5 | 456.2 | 164.3 | 270.9 | 53.8 | 75.6 | 99.0 | 292.8 | 213.7 |
| 23 | | 307.1 | 147.0 | 83.2 | 50.0 | 446.9 | 168.8 | 315.9 | 56.3 | 315.9 | 90.0 | 344.3 | 211.4 |
| 25 | | 359.5 | 161.3 | 53.8 | 81.2 | 428.7 | 174.4 | 364.5 | 42.5 | 245.4 | 90.0 | 364.5 | 215.1 |
| 28 | | 383.6 | 166.6 | 56.3 | 67.4 | 415.2 | 163.3 | 292.6 | 56.3 | 270.9 | 90.9 | 340.6 | 209.4 |
| 31 | | 310.0 | 159.3 | 40.5 | 50.0 | 307.1 | 159.3 | 296.2 | 50.6 | 258.6 | 83.2 | 307.1 | 183.8 |
| 34 | | 442.2 | 182.8 | 71.5 | 78.4 | 428.7 | 249.6 | 325.1 | 75.7 | 343.2 | 147.9 | 329.3 | 243.1 |
| 38 | | 470.6 | 325.1 | 252.9 | 159.3 | 428.7 | 380.9 | 500.0 | 142.1 | 428.7 | 224.0 | 344.3 | 332.4 |
| 42 | | 726.0 | 445.5 | 289.0 | 171.5 | 450.0 | 450.0 | 500.0 | 224.0 | 428.7 | 272.0 | 428.7 | 398.7 |
| 46 | | 665.5 | 500.0 | 289.0 | 168.8 | | 496.1 | 406.1 | 196.9 | 406.1 | 256.0 | 470.6 | 385.5 |
| 52 | | 786.5 | 864.0 | 325.1 | 168.8 | | 496.1 | 406.1 | 196.9 | 442.2 | 256.0 | 523.7 | 446.5 |

TABLE 2
Tumor growth in secondary tumors

a) Tumor growth in secondary tumors (Control IgG Group)

| Day post | Control Group (Mice #1-10) | | | | | | | | | | | |
|---|---|---|---|---|---|---|---|---|---|---|---|---|
| tumor inj. | ID# | 1686 | 1670 | 1786 | 1643 | 1518 | 1517 | 6683 | 1644 | 1520 | 1557 | Mean |
| 16 | | 0.0 | 0.0 | 0.0 | 0.0 | 0.0 | 0.0 | 0.0 | 0.0 | 0.0 | 0.0 | 0.0 |
| 18 | | 0.0 | 0.0 | 0.0 | 0.0 | 0.0 | 0.0 | 0.0 | 0.0 | 0.0 | 0.0 | 0.0 |
| 15 | | 4.0 | 0.0 | 0.0 | 0.0 | 12.2 | 13.5 | 0.0 | 18.4 | 18.4 | 4.0 | 7.0 |
| 22 | | 11.3 | 0.0 | 50.0 | 50.0 | 13.5 | 63.4 | 6.3 | 35.4 | 99.0 | 0.0 | 32.9 |
| 25 | | 42.5 | 0.0 | 63.5 | 83.2 | 146.3 | 196.0 | 40.0 | 67.5 | 116.2 | 24.0 | 77.9 |
| 28 | | 40.0 | 0.0 | 216.8 | 171.5 | 208.0 | 364.5 | 90.8 | 171.5 | 240.0 | 36.0 | 153.9 |
| 31 | | 275.7 | 0.0 | 252.9 | 174.4 | 451.3 | 695.8 | 208.0 | 544.5 | 307.1 | 35.4 | 294.5 |
| 34 | | 289.0 | 0.0 | 426.6 | 406.1 | 451.3 | 760.4 | 353.4 | 496.1 | 470.6 | 45.6 | 369.9 |
| 38 | | 1078.0 | 0.0 | 720.0 | 695.8 | 1372.0 | 1176.0 | 428.7 | 1008.0 | 859.4 | 171.5 | 750.9 |

b) Tumor growth in secondary tumors (OX86 Group)

| Days post | OX86 (Mice# 1-10) | | | | | | | | | | | | |
|---|---|---|---|---|---|---|---|---|---|---|---|---|---|
| tumor inj. | ID# | 1668 | 1577 | 1658 | 1776 | 1521 | 1576 | 1511 | 1649 | 1667 | 1789 | 1793 | Mean |
| 16 | | 0.0 | 0.0 | 0.0 | 0.0 | 0.0 | 0.0 | 0.0 | 0.0 | 0.0 | 0.0 | 0.0 | 0.0 |
| 18 | | 0.0 | 0.0 | 0.0 | 0.0 | 0.0 | 0.0 | 0.0 | 0.0 | 0.0 | 0.0 | 0.0 | 0.0 |
| 15 | | 0.0 | 0.0 | 0.0 | 0.0 | 0.0 | 0.0 | 0.0 | 0.0 | 0.0 | 0.0 | 0.0 | 0.0 |
| 22 | | 13.5 | 13.5 | 0.0 | 6.3 | 6.3 | 28.0 | 11.3 | 6.3 | 18.4 | 18.4 | 40.0 | 14.7 |
| 25 | | 16.7 | 22.7 | 0.0 | 10.9 | 55.7 | 50.8 | 28.0 | 44.0 | 117.0 | 45.6 | 57.6 | 40.8 |
| 28 | | 28.0 | 27.0 | 0.0 | 52.0 | 90.0 | 50.7 | 44.2 | 40.5 | 77.1 | 62.5 | 45.6 | 47.0 |
| 31 | | 15.8 | 23.1 | 0.0 | 15.8 | 62.5 | 75.0 | 21.4 | 24.5 | 171.5 | 54.0 | 159.3 | 56.6 |
| 34 | | 27.6 | 32.0 | 0.0 | 53.8 | 183.8 | 81.3 | 65.0 | 32.5 | 364.5 | 114.4 | 313.6 | 115.3 |
| 38 | | 108.0 | 196.9 | 0.0 | 140.6 | 324.0 | 303.8 | 137.3 | 90.0 | 1406.3 | 240.0 | 694.3 | 331.0 |
| 42 | | 108.0 | 196.9 | 0.0 | 196.9 | 523.7 | 303.8 | 171.5 | 108.0 | 1470.0 | 283.5 | 980.0 | 394.7 |
| 44 | | 108.0 | 171.5 | 0.0 | 196.9 | 523.7 | 605.0 | 324.0 | 210.9 | 1470.0 | 344.3 | 1127.0 | 461.9 |

c) Tumor growth in secondary tumors (Radiation Group)

| Days Post | Radiation | | | | | | | | |
|---|---|---|---|---|---|---|---|---|---|
| tumor inj. | ID# | 1693 | 1790 | 1503 | 1785 | 1504 | 1523 | 1664 | Mean |
| 16 | | 0.0 | 0.0 | 0.0 | 0.0 | 0.0 | 0.0 | 0.0 | 0.0 |
| 18 | | 0.0 | 0.0 | 0.0 | 0.0 | 0.0 | 0.0 | 0.0 | 0.0 |
| 21 | | 9.4 | 9.4 | 4.0 | 0.0 | 18.4 | 0.0 | 0.0 | 5.9 |
| 23 | | 32.0 | 28.0 | 6.3 | 161.8 | 43.8 | 45.6 | 56.3 | 53.4 |
| 25 | | 104.4 | 53.0 | 10.0 | 278.4 | 48.6 | 108.0 | 191.1 | 113.4 |
| 28 | | 210.9 | 50.6 | 22.7 | 406.1 | 32.0 | 100.8 | 249.6 | 153.3 |
| 31 | | 256.0 | 93.8 | 21.4 | 792.0 | 142.8 | 376.2 | 288.0 | 281.5 |
| 34 | | 317.5 | 171.5 | 65.0 | 847.0 | 171.6 | 384.8 | 324.0 | 325.9 |
| 38 | | 475.0 | 726.0 | 182.8 | 1687.5 | 1372.0 | 475.0 | 500.0 | 774.0 |
| 42 | | 1734.6 | 847.0 | 182.8 | 1912.5 | 364.5 | 665.5 | 1372.0 | 1011.3 |

d) Tumor growth in secondary tumors (Radiation + OX86 Group)

| Days Post | Radiation + OX86 | | | | | | | | | | | | |
|---|---|---|---|---|---|---|---|---|---|---|---|---|---|
| Tumor Inj. | ID# | 1525 | 1694 | 1542 | 1501 | 1671 | 1653 | 1791 | 1673 | 1792 | 1641 | 1505 | Mean |
| 16 | | 0.0 | 0.0 | 0.0 | 0.0 | 0.0 | 0.0 | 0.0 | 0.0 | 0.0 | 0.0 | 0.0 | 0.0 |
| 18 | | 0.0 | 0.0 | 0.0 | 0.0 | 0.0 | 0.0 | 0.0 | 0.0 | 0.0 | 0.0 | 0.0 | 0.0 |
| 21 | | 4.0 | 4.0 | 4.0 | 20.8 | 4.0 | 30.4 | 27.0 | 4.0 | 122.5 | 4.0 | 4.0 | 20.8 |
| 23 | | 13.5 | 15.8 | 4.0 | 18.4 | 6.0 | 13.5 | 35.4 | 21.4 | 35.4 | 18.4 | 11.3 | 17.6 |
| 25 | | 28.0 | 35.4 | 13.5 | 45.6 | 6.0 | 35.4 | 45.6 | 43.5 | 45.6 | 29.6 | 20.3 | 31.7 |
| 28 | | 24.5 | 47.5 | 13.5 | 28.0 | 10.9 | 35.4 | 62.5 | 45.6 | 43.5 | 28.0 | 24.5 | 33.1 |
| 31 | | 24.5 | 68.1 | 13.5 | 50.0 | 7.8 | 20.3 | 50.0 | 21.4 | 21.4 | 18.0 | 12.5 | 28.0 |
| 34 | | 75.6 | 126.8 | 15.8 | 87.7 | 23.4 | 135.0 | 113.4 | 48.6 | 87.7 | 25.9 | 32.8 | 70.2 |
| 38 | | 83.2 | 406.1 | 68.8 | 256.0 | 18.4 | 81.0 | 117.0 | 147.0 | 152.1 | 99.0 | 147.0 | 143.2 |
| 42 | | 116.2 | 405.0 | 83.2 | 544.5 | 137.3 | 117.0 | 208.0 | 210.9 | 210.9 | 108.0 | 92.5 | 203.1 |
| 44 | | 147.0 | 551.3 | 90.0 | 171.5 | 0.0 | 117.0 | 182.8 | 299.8 | 256.0 | 171.5 | 113.4 | 190.9 |
| 46 | | 196.0 | 661.5 | 171.5 | 171.5 | 0.0 | 224.0 | 183.8 | 307.1 | 256.0 | 171.5 | 183.8 | 229.7 |
| 52 | | 267.2 | 0.0 | 126.8 | | | 182.8 | 252.9 | 976.6 | 256.0 | 364.5 | 500.0 | 325.2 |

TABLE 3
Statistics between groups in primary tumors (Multiple T test)

| | | | | | | | |
|---|---|---|---|---|---|---|---|
| Days post | | | | | SE of | | |
| tumor inj. | P value | Mean1 | Mean2 | Difference | difference | t ratio | df |

a) Control IgG vs OX86 with radiation in primary tumors

| | | | | | | | |
|---|---|---|---|---|---|---|---|
| 16 | 0.241586 | 28.785 | 44.6591 | −15.8741 | 13.1326 | 1.20875 | 19 |
| 18 | 0.535301 | 93.4233 | 114.397 | −20.9735 | 33.2174 | 0.631402 | 19 |
| 19 | 0.052126 | 118.74 | 213.731 | −94.9908 | 45.8465 | 2.07193 | 19 |
| 22 | 0.182677 | 139.508 | 211.38 | −71.8713 | 51.9644 | 1.38309 | 19 |
| 25 | 0.634006 | 248.063 | 215.075 | 32.9872 | 68.1733 | 0.483873 | 19 |
| 28 | 0.0791783 | 358.057 | 209.42 | 148.637 | 80.1268 | 1.85502 | 19 |
| 31 | 0.0037449 | 565.198 | 183.794 | 381.403 | 115.492 | 3.30242 | 19 |
| 34 | 0.0025038 | 761.123 | 243.129 | 517.994 | 148.828 | 3.48049 | 19 |
| 38 | 0.0006837 | 1064.81 | 332.405 | 732.402 | 180.847 | 4.04985 | 19 |

b) OX86 vs OX86 with radiation in primary tumor

| | | | | | | | |
|---|---|---|---|---|---|---|---|
| 16 | 0.232435 | 28.5602 | 44.6591 | −16.0988 | 13.0514 | 1.23349 | 19 |
| 18 | 0.207657 | 73.1913 | 114.397 | −41.2055 | 31.5881 | 1.30446 | 19 |
| 19 | 0.101442 | 136.279 | 213.731 | −77.4517 | 44.9965 | 1.72128 | 19 |
| 22 | 0.186567 | 144.695 | 211.38 | −66.6843 | 48.6639 | 1.3703 | 19 |
| 25 | 0.405769 | 168.277 | 215.075 | −46.7987 | 55.0405 | 0.850258 | 19 |
| 28 | 0.887558 | 201.514 | 209.42 | −7.90657 | 55.1728 | 0.143306 | 19 |
| 31 | 0.941993 | 187.492 | 183.794 | 3.69808 | 50.1548 | 0.0737334 | 19 |
| 34 | 0.410032 | 304.771 | 243.129 | 61.6417 | 73.1721 | 0.842421 | 19 |
| 38 | 0.0174422 | 729.925 | 332.405 | 397.521 | 152.668 | 2.60383 | 19 |
| 42 | 0.0282547 | 931.118 | 398.67 | 532.448 | 223.202 | 2.3855 | 18 |
| 46 | 0.0022181 | 930.348 | 385.51 | 544.839 | 147.962 | 3.68229 | 15 |

c) Radiation vs OX86 with radiation in primary tumors

| | | | | | | | |
|---|---|---|---|---|---|---|---|
| 16 | 0.525469 | 56.7875 | 44.6591 | 12.1284 | 18.6843 | 0.649121 | 16 |
| 18 | 0.95435 | 116.981 | 114.397 | 2.5839 | 44.4357 | 0.058149 | 16 |
| 19 | 0.967608 | 211.352 | 213.731 | −2.37903 | 57.676 | 0.041248 | 16 |
| 22 | 0.962716 | 214.377 | 211.38 | 2.99751 | 63.1283 | 0.047483 | 16 |
| 25 | 0.313706 | 284.486 | 215.075 | 69.4107 | 66.7275 | 1.04021 | 16 |
| 28 | 0.194251 | 299.477 | 209.42 | 90.0565 | 66.464 | 1.35497 | 16 |
| 31 | 0.131256 | 283.691 | 183.794 | 99.897 | 62.8037 | 1.59062 | 16 |
| 34 | 0.016502 | 450.835 | 243.129 | 207.706 | 77.5636 | 2.67788 | 16 |
| 38 | 0.000159 | 759.268 | 332.405 | 426.863 | 87.0504 | 4.90363 | 16 |
| 42 | 3.34E−05 | 1015.37 | 398.67 | 616.696 | 108.364 | 5.69098 | 16 |

d) Control IgG vs OX86 in primary tumors

| | | | | | | | |
|---|---|---|---|---|---|---|---|
| 16 | 0.9781 | 28.785 | 28.5602 | 0.22475 | 8.07445 | 0.027835 | 18 |
| 18 | 0.382332 | 93.4233 | 73.1913 | 20.232 | 22.5926 | 0.895513 | 18 |
| 19 | 0.604149 | 118.74 | 136.279 | −17.5391 | 33.2365 | 0.527706 | 18 |
| 22 | 0.88939 | 139.508 | 144.695 | −5.18705 | 36.7724 | 0.141058 | 18 |
| 25 | 0.218309 | 248.063 | 168.277 | 79.7859 | 62.547 | 1.27561 | 18 |
| 28 | 0.064919 | 358.057 | 201.514 | 156.543 | 79.626 | 1.96598 | 18 |
| 31 | 0.005835 | 565.198 | 187.492 | 377.705 | 120.819 | 3.12621 | 18 |
| 34 | 0.011183 | 761.123 | 304.771 | 456.353 | 161.456 | 2.82648 | 18 |
| 38 | 0.183291 | 1064.81 | 729.925 | 334.881 | 241.972 | 1.38396 | 18 |
| e) Control IgG vs radiation in primary tumors | | | | | | | |
| 16 | 0.070832 | 28.785 | 56.7875 | −28.0025 | 14.4009 | 1.9445 | 15 |
| 18 | 0.527152 | 93.4233 | 116.981 | −23.5574 | 36.3872 | 0.647409 | 15 |
| 19 | 0.057862 | 118.74 | 211.352 | −92.6117 | 45.0942 | 2.05374 | 15 |
| 22 | 0.160448 | 139.508 | 214.377 | −74.8688 | 50.701 | 1.47667 | 15 |
| 25 | 0.638008 | 248.063 | 284.486 | −36.4234 | 75.8494 | 0.480207 | 15 |
| 28 | 0.551132 | 358.057 | 299.477 | 58.5802 | 96.0674 | 0.609782 | 15 |
| 31 | 0.07463 | 565.198 | 283.691 | 281.506 | 146.928 | 1.91595 | 15 |
| 34 | 0.122896 | 761.123 | 450.835 | 310.288 | 189.801 | 1.63481 | 15 |
| 38 | 0.216813 | 1064.81 | 759.268 | 305.538 | 236.976 | 1.28932 | 15 |

f) OX86 vs radiation Primary tumors

| | | | | | SE of | | |
|---|---|---|---|---|---|---|---|
| Days post | P value | Mean1 | Mean2 | Difference | difference | t ratio | df |
| 16 | 0.066784 | 28.5602 | 56.7875 | −28.2272 | 14.2815 | 1.97649 | 15 |
| 18 | 0.21689 | 73.1913 | 116.981 | −43.7894 | 33.9691 | 1.2891 | 15 |
| 19 | 0.106327 | 136.279 | 211.352 | −75.0726 | 43.6928 | 1.71819 | 15 |
| 22 | 0.143297 | 144.695 | 214.377 | −69.6818 | 45.1157 | 1.54451 | 15 |
| 25 | 0.055925 | 168.277 | 284.486 | −116.209 | 56.0871 | 2.07194 | 15 |
| 28 | 0.132337 | 201.514 | 299.477 | −97.963 | 61.552 | 1.59155 | 15 |
| 31 | 0.156021 | 187.492 | 283.691 | −96.1989 | 64.4082 | 1.49358 | 15 |
| 34 | 0.143661 | 304.771 | 450.835 | −146.064 | 94.6624 | 1.543 | 15 |
| 38 | 0.886729 | 729.925 | 759.268 | −29.3424 | 202.519 | 0.144887 | 15 |
| 42 | 0.77874 | 931.118 | 1015.37 | −84.248 | 294.126 | 0.286435 | 14 |

TABLE 4
Statistics between groups in secondary tumors (Multiple T test)

| | | | | | | | |
|---|---|---|---|---|---|---|---|
| Days post | | | | | SE of | | |
| tumor inj. | P value | Mean1 | Mean2 | Difference | difference | t ratio | df |

a) Control IgG vs OX86 with radiation in secondary tumors

| | | | | | | | |
|---|---|---|---|---|---|---|---|
| 16 | | | | | | | |
| 18 | 0.243806 | 7.04375 | 20.7886 | −13.7449 | 11.4268 | 1.20286 | 19 |
| 19 | 0.154904 | 32.8812 | 17.5511 | 15.3301 | 10.3486 | 1.48136 | 19 |
| 22 | 0.021618 | 77.9175 | 31.6773 | 46.2402 | 18.4763 | 2.50267 | 19 |
| 25 | 0.002209 | 153.908 | 33.0886 | 120.819 | 34.1719 | 3.53563 | 19 |
| 28 | 0.000674 | 294.493 | 27.9517 | 266.542 | 65.7129 | 4.05615 | 19 |
| 31 | 0.000294 | 369.91 | 70.246 | 299.664 | 67.7971 | 4.42001 | 19 |
| 34 | 0.000287 | 750.931 | 143.231 | 607.701 | 137.145 | 4.4311 | 19 |
| 38 | 0.000471 | 1079.94 | 203.052 | 876.892 | 208.122 | 4.21336 | 19 |

b) OX86 vs OX86 with radiation in secondary tumors

| | | | | | | | |
|---|---|---|---|---|---|---|---|
| | 0.232435 | 28.5602 | 44.6591 | −16.0988 | 13.0514 | 1.23349 | 19 |
| 16 | 0.207657 | 73.1913 | 114.397 | −41.2055 | 31.5881 | 1.30446 | 19 |
| 18 | 0.101442 | 136.279 | 213.731 | −77.4517 | 44.9965 | 1.72128 | 19 |
| 19 | 0.186567 | 144.695 | 211.38 | −66.6843 | 48.6639 | 1.3703 | 19 |
| 22 | 0.405769 | 168.277 | 215.075 | −46.7987 | 55.0405 | 0.850258 | 19 |
| 25 | 0.887558 | 201.514 | 209.42 | −7.90657 | 55.1728 | 0.143306 | 19 |
| 28 | 0.941993 | 187.492 | 183.794 | 3.69808 | 50.1548 | 0.073733 | 19 |
| 31 | 0.410032 | 304.771 | 243.129 | 61.6417 | 73.1721 | 0.842421 | 19 |
| 34 | 0.017442 | 729.925 | 332.405 | 397.521 | 152.668 | 2.60383 | 19 |
| 38 | 0.028255 | 931.118 | 398.67 | 532.448 | 223.202 | 2.3855 | 18 |
| 42 | 0.002218 | 930.348 | 385.51 | 544.839 | 147.962 | 3.68229 | 15 |

c) Radiation vs OX86 with radiation in secondary tumors

| | | | | | | | |
|---|---|---|---|---|---|---|---|
| | 0.525469 | 56.7875 | 44.6591 | 12.1284 | 18.6843 | 0.649121 | 16 |
| 16 | 0.95435 | 116.981 | 114.397 | 2.5839 | 44.4357 | 0.058149 | 16 |
| 18 | 0.967608 | 211.352 | 213.731 | −2.37903 | 57.676 | 0.041248 | 16 |
| 19 | 0.962716 | 214.377 | 211.38 | 2.99751 | 63.1283 | 0.047483 | 16 |
| 22 | 0.313706 | 284.486 | 215.075 | 69.4107 | 66.7275 | 1.04021 | 16 |
| 25 | 0.194251 | 299.477 | 209.42 | 90.0565 | 66.464 | 1.35497 | 16 |
| 28 | 0.131256 | 283.691 | 183.794 | 99.897 | 62.8037 | 1.59062 | 16 |
| 31 | 0.016502 | 450.835 | 243.129 | 207.706 | 77.5636 | 2.67788 | 16 |
| 34 | 0.000159 | 759.268 | 332.405 | 426.863 | 87.0504 | 4.90363 | 16 |
| 38 | 3.34E−05 | 1015.37 | 398.67 | 616.696 | 108.364 | 5.69098 | 16 |

d) Control IgG group VS Anti 0X40 alone in secondary tumors. (P value < 0.05)

| | | | | | | | |
|---|---|---|---|---|---|---|---|
| 16 | | | | | | | |
| 18 | | | | | | | |
| 19 | 0.007065 | 7.04375 | 0 | 7.04375 | 2.33348 | 3.01855 | 19 |
| 22 | 0.098574 | 32.8812 | 14.7045 | 18.1767 | 10.4645 | 1.73699 | 19 |
| 25 | 0.087334 | 77.9175 | 40.8134 | 37.1041 | 20.5834 | 1.80262 | 19 |
| 28 | 0.006174 | 153.908 | 47.0471 | 106.861 | 34.7034 | 3.07926 | 19 |
| 31 | 0.002404 | 294.493 | 56.6209 | 237.872 | 67.9942 | 3.49842 | 19 |
| 34 | 0.003637 | 369.91 | 115.298 | 254.612 | 76.7966 | 3.3154 | 19 |
| 38 | 0.034072 | 750.931 | 331.011 | 419.92 | 183.879 | 2.28367 | 19 |
| 42 | 0.012152 | 1079.94 | 394.744 | 685.199 | 247.223 | 2.77158 | 19 |

e) Control IgG vs radiation secondary tumors

| | | | | | | | |
|---|---|---|---|---|---|---|---|
| 16 | 0.070832 | 28.785 | 56.7875 | −28.0025 | 14.4009 | 1.9445 | 15 |
| 18 | 0.527152 | 93.4233 | 116.981 | −23.5574 | 36.3872 | 0.647409 | 15 |
| 19 | 0.057862 | 118.74 | 211.352 | −92.6117 | 45.0942 | 2.05374 | 15 |
| 22 | 0.160448 | 139.508 | 214.377 | −74.8688 | 50.701 | 1.47667 | 15 |
| 25 | 0.638008 | 248.063 | 284.486 | −36.4234 | 75.8494 | 0.480207 | 15 |
| 28 | 0.551132 | 358.057 | 299.477 | 58.5802 | 96.0674 | 0.609782 | 15 |
| 31 | 0.07463 | 565.198 | 283.691 | 281.506 | 146.928 | 1.91595 | 15 |
| 34 | 0.122896 | 761.123 | 450.835 | 310.288 | 189.801 | 1.63481 | 15 |
| 38 | 0.216813 | 1064.81 | 759.268 | 305.538 | 236.976 | 1.28932 | 15 |

f) OX86 vs radiation secondary tumors

| | | | | | SE of | | |
|---|---|---|---|---|---|---|---|
| Days post | P value | Mean1 | Mean2 | Difference | difference | t ratio | df |
| 16 | | | | | | | |
| 18 | | | | | | | |
| 19 | 0.011227 | 0 | 5.875 | −5.875 | 2.05062 | 2.86498 | 16 |
| 22 | 0.024236 | 14.7045 | 53.3826 | −38.6781 | 15.5437 | 2.48834 | 16 |
| 25 | 0.028312 | 40.8134 | 113.359 | −72.546 | 30.0938 | 2.41066 | 16 |
| 28 | 0.025697 | 47.0471 | 153.25 | −106.203 | 43.1867 | 2.45916 | 16 |
| 31 | 0.011614 | 56.6209 | 281.47 | −224.849 | 78.9323 | 2.84863 | 16 |
| 34 | 0.030526 | 115.298 | 325.902 | −210.604 | 88.758 | 2.37279 | 16 |
| 38 | 0.064374 | 331.011 | 774.045 | −443.033 | 223.015 | 1.98657 | 16 |
| 42 | 0.031781 | 394.744 | 1011.27 | −616.524 | 262.077 | 2.35245 | 16 |

TABLE 5
Number of lung metastases in mice at day 32, 48 hours post final dose of OX86

| Number of | | | | | | |
|---|---|---|---|---|---|---|
| metastases in | | | | | | |
| lungs | Mouse#1 | Mouse#2 | Mouse#3 | Mouse#4 | Mouse#5 | Mouse#6 |
| Ctrl IgG | 41 | 22 | 35 | 30 | 60 | 43 |
| _{α}-OX40 | 3 | 35 | 10 | 2 | 12 | 52 |
| XRT 12Gyx3 | 24 | 31 | 40 | 6 | 37 | 48 |
| XRT3*12Gy + _{α}-OX40 | 0 | 16 | 0 | 20 | 3 | 0 |

| Groups | Summary | P value |
|---|---|---|
| Ctrl IgG vs αOX40 | Ns | 0.1261 |
| Ctrl IgG vs XRT | Ns | 0.4030 |
| 12Gyx3 | | |
| Ctrl IgG vs αOX40 + | ** | 0.0075 |
| XRT 12Gyx3 | | |
| αOX40 vs XRT 12Gyx3 | Ns | 0.4030 |
| αOX40 vs αOX40 + | Ns | 0.4030 |
| XRT 12Gyx3 | | |
| XRT 12Gyx3 vs αOX40 + | * | 0.0459 |
| XRT 12Gyx3 | | |

TABLE 6
Percentage of CD4 T cells in primary tumors
at day 32, 48 hrs post final dose of OX86

| % of CD4 | Mouse#1 | Mouse#2 | Mouse#3 |
|---|---|---|---|
| Ctrl IgG | 14.4 | 6.88 | 15.5 |
| _{α}-OX40 | 5.05 | 9.59 | 11 |
| XRT 12Gyx3 | 5.87 | 17.2 | 10.8 |
| XRT3*12Gy + _{α}-OX40 | 16.9 | 25.9 | 14.4 |

TABLE 7
Percentage of CD4 T cells in secondary tumors
at day 32, 48 hrs post final dose of OX86

| % of CD4 | Mouse#1 | Mouse#2 | Mouse#3 |
|---|---|---|---|
| Ctrl IgG | 10.9 | 1.91 | 2.93 |
| _{α}-OX40 | 7.45 | 7.71 | 11.7 |
| XRT 12Gyx3 | 2.65 | 4.3 | 9.51 |
| XRT3*12Gy + _{α}-OX40 | 5.69 | 21.2 | 18.3 |

TABLE 8
Percentage of CD8 T cells in primary tumors
at day 32, 48 hrs post final dose of OX86

| % of CD8 | Mouse#1 | Mouse#2 | Mouse#3 |
|---|---|---|---|
| Ctrl IgG | 1.46 | 0.34 | 0 |
| _{α}-OX40 | 3.03 | 6.16 | 1.34 |
| XRT 12Gyx3 | 3.03 | 0.59 | 2.91 |
| XRT3*12Gy + _{α}-OX40 | 5.12 | 3.35 | 11 |

TABLE 9
Percentage of CD8 T cells in secondar tumors
at day 32, 48 hrs post final dose of OX86

| % of CD8 | Mouse#1 | Mouse#2 | Mouse#3 |
|---|---|---|---|
| Ctrl IgG | 0.41 | 2.93 | 1.82 |
| _{α}-OX40 | 2.54 | 6.88 | 4.91 |
| XRT 12Gyx3 | 7.89 | 2.31 | 1.43 |
| XRT3*12Gy + _{α}-OX40 | 5.12 | 3.35 | 11 |

TABLE 10
Percentage of dendritic cells in spleens
48 hours following radiation

| | Mouse#1 | Mouse#2 | Mouse#3 | Mouse#4 |
|---|---|---|---|---|
| DCs No XRT | 3.99 | 5.34 | 5.33 | |
| DCs XRT | 5.94 | 11.2 | 9.57 | 6.44 |
| CD103 + DCs No XRT | 0.03 | 0.09 | 0.09 | |
| CD103 + DCs XRT | 0.62 | 2.32 | 1.44 | 0.8 |

| Groups | Summary | P value |
|---|---|---|
| Ctrl IgG vs XRT | Ns | 0.0651 |
| Ctrl IgG vs XRT (CD | * | 0.0484 |
| 103 + DC) | | |

TABLE 11
Percentage of CD4 T cells in spleens 48 hours following radiation

| | mouse#1 | Mouse#2 | Mouse#3 | Mouse#4 |
|---|---|---|---|---|
| No Treatment | 4.45 | 5.85 | 5.8 | |
| Radiation | 7.45 | 10.5 | 8.45 | 7.41 |

TABLE 12
Percentage of CD8 T cells in spleens 48 hours following radiation

| | mouse#1 | Mouse#2 | Mouse#3 | Mouse#4 |
|---|---|---|---|---|
| No Treatment | 0.47 | 1.6 | 1.5 | |
| Radiation | 1.89 | 2.7 | 1.72 | 1.61 |

TABLE 13
Percentage of dendritic cells in spleens 7 days following radiation

| | mouse#1 | Mouse#2 | Mouse#3 | Mouse#4 |
|---|---|---|---|---|
| DCs No XRT | 7.13 | 5.63 | 5.3 | |
| DCs XRT | 6.66 | 8.49 | 6.76 | 9.31 |
| CD103 + DCs No XRT | 0.7 | 0.35 | 0.7 | |
| CD103 + DCs XRT | 0.44 | 1 | 0.4 | 1.35 |

TABLE 14
Percentage of CD4 T cells in tumors 7 days following radiation

| | mouse#1 | Mouse#2 | Mouse#3 | Mouse#4 |
|---|---|---|---|---|
| No Treatment | 5.99 | 8.6 | 6.3 | |
| Radiation | 17.2 | 15.1 | 15.1 | 11.8 |

TABLE 15
Percentage of CD4 T cells in spleens 7 days following radiation

| | mouse#1 | Mouse#2 | Mouse#3 | Mouse#4 |
|---|---|---|---|---|
| No Treatment | 1.95 | 2.63 | 2.51 | |
| Radiation | 2.69 | 4.21 | 3.22 | 4.52 |

TABLE 16
OX40L expression on neutrophils in primary tumors at
day 32, 48 hrs post the final dose of OX86 or isotype

| | mouse#1 | Mouse#2 | Mouse#3 |
|---|---|---|---|
| CTRL. IgG | 1.49 | 2.94 | 1.49 |
| anti-OX40 | 0.52 | 0 | 2.27 |
| XRT | 1.3 | 10.7 | 0.68 |
| anti-OX40 + XRT | 2.61 | 6.76 | 46.3 |

TABLE 17
OX40L expression on macrophages in primary tumors at
day 32, 48 hrs post the final dose of 0X86 or isotype

| | mouse#1 | Mouse#2 | Mouse#3 |
|---|---|---|---|
| CTRL. IgG | 12.1 | 12.1 | 3.49 |
| anti-OX40 | 4.25 | 1.45 | 6.86 |
| XRT | 4.47 | 7.95 | 7.87 |
| anti-OX40 + XRT | 6.98 | 5.11 | 33 |

1.-95. (canceled)

96. A method of treating human cancer, the method comprising:
(i) administering an anti-human OX40 antigen binding protein comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and,
(ii) administering radiotherapy;
wherein a therapeutically effective amount of the anti-human OX40 antigen binding protein and the radiotherapy is administered.

97. The method of claim 96, wherein the radiotherapy is selected from the group consisting of systemic radiation therapy, external beam radiation therapy, image-guided radiation therapy, tomotherapy, stereotactic radio surgery, stereotactic body radiation therapy, and proton therapy.

98. The method of claim 96, wherein the cancer is a solid tumor.

99. The method of claim 98, wherein the solid tumor is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

100. The method of claim 96, wherein the anti-human OX40 antigen binding protein is a monoclonal antibody.

101. The method according to claim 100, wherein the anti-human OX40 monoclonal antibody is a human monoclonal antibody or a humanized monoclonal antibody.

102. The method according to claim 100, wherein the anti-human OX40 monoclonal antibody is an IgG1 antibody isotype, an IgG4 antibody isotype, or a variant thereof.

103. The method of claim 96, wherein the human cancer is anti-PD-1 resistant.

104. The method of claim 96, wherein the anti-human OX40 antigen binding protein is administered systemically or intratumorally.

105. The method of claim 96, wherein the anti-human OX40 antigen binding protein comprises:
(i) a light chain variable region comprising an amino acid sequence with at least 90%, 91%, 92%, 93%, 94%, 95%, 96%, 97%, 98%, 99%, or 100% sequence identity to the amino acid sequence as set forth in SEQ ID NO: 11; and
(ii) a heavy chain variable region comprising an amino acid sequence with at least 90%, 91%, 92%, 93%, 94%, 95%, 96%, 97%, 98%, 99%, or 100% sequence identity to the amino acid sequence as set forth in SEQ ID NO: 5.

106. The method of claim 105, wherein the anti-human OX40 antigen binding protein comprises a heavy chain variable region comprising the amino acid sequence set forth in SEQ ID NO:5 and a light chain variable region comprising the amino acid sequence set forth in SEQ ID NO:11.

107. The method of claim 96, wherein the anti-human OX40 antigen binding protein comprises a heavy chain comprising an amino acid sequence with at least 90%, 91%, 92%, 93%, 94%, 95%, 96%, 97%, 98%, 99%, or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99%, or 100% sequence identity to the amino acid sequence as set forth in SEQ ID NO:49.

108. A method of treating a human solid tumor, the method comprising:
(i) administering an anti-human OX40 monoclonal antibody, the antibody comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and, (f) a light chain variable region CDR3 comprising the amino acid sequence of SEQ ID NO:9;
(ii) administering radiotherapy; and,
(iii) administering an anti-PD-1 monoclonal antibody;
wherein a therapeutically effective amount of the anti-human OX40 monoclonal antibody, the radiotherapy, and the anti-PD-1 monoclonal antibody is administered.

109. The method of claim 108, wherein the anti-PD-1 monoclonal antibody is pembrolizumab, or an antibody comprising 90%, 91%, 92%, 93%, 94%, 95%, 96%, 97%, 98%, 99%, or 100% sequence identity to each of heavy chain SEQ ID NO:50 and light chain SEQ ID NO:51.

110. The method of claim 108, wherein the anti-PD-1 monoclonal antibody is nivolumab, or an antibody having 90%, 91%, 92%, 93%, 94%, 95%, 96%, 97%, 98%, 99%, or 100% sequence identity to each of heavy chain SEQ ID NO:98 and light chain SEQ ID NO:99.

111. The method of claim 108, the method further comprising the administration of at least one additional anti-neoplastic agent.

112. The method of claim 108, wherein the human solid tumor is selected from the group consisting of melanoma, lung cancer, kidney cancer, breast cancer, head and neck cancer, colon cancer, ovarian cancer, pancreatic cancer, liver cancer, prostate cancer, bladder cancer, and gastric cancer.

113. The method of claim 108, wherein the human solid tumor is anti-PD-1 resistant.

114. A method for reducing tumor size in a human in need thereof, the method comprising: (i) administering an anti-human OX40 monoclonal antibody; (ii) administering radiotherapy; and, (iii) administering an anti-PD-1 monoclonal antibody.

115. The method of claim 114, wherein the anti-human OX40 monoclonal antibody comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and, (f) a light chain variable region CDR3 comprising the amino acid sequence of SEQ ID NO:9.

116. The method of claim 115, wherein the tumor is a solid tumor selected from the group consisting of melanoma, lung cancer, kidney cancer, breast cancer, head and neck cancer, colon cancer, ovarian cancer, pancreatic cancer, liver cancer, prostate cancer, bladder cancer, and gastric cancer.

117. The method of claim 116, wherein the anti-PD-1 monoclonal antibody is selected from the group consisting of: pembrolizumab; an antibody comprising 90%, 91%, 92%, 93%, 94%, 95%, 96%, 97%, 98%, 99%, or 100% sequence identity to each of heavy chain SEQ ID NO:50 and light chain SEQ ID NO:51; nivolumab; and, an antibody having 90%, 91%, 92%, 93%, 94%, 95%, 96%, 97%, 98%, 99%, or 100% sequence identity to each of heavy chain SEQ ID NO:98 and light chain SEQ ID NO:99.


SEQUENCE LISTING (107 sequences)

SEQ ID NO: 1 - protein, 5 aa, Mus sp.
DYSMH

SEQ ID NO: 2 - protein, 17 aa, Mus sp.
WINTETGEPTYADDFKG

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, Artificial Sequence
  source: /note=“Description of Artificial Sequence: Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, Artificial Sequence
  source: /note=“Description of Artificial Sequence: Synthetic polynucleotide”
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, Artificial Sequence
  source: /note=“Description of Artificial Sequence: Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, Artificial Sequence
  source: /note=“Description of Artificial Sequence: Synthetic polynucleotide”
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, Artificial Sequence
  source: /note=“Description of Artificial Sequence: Synthetic polypeptide”
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, Artificial Sequence
  source: /note=“Description of Artificial Sequence: Synthetic polynucleotide”
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, Artificial Sequence
  source: /note=“Description of Artificial Sequence: Synthetic polypeptide”
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, Artificial Sequence
  source: /note=“Description of Artificial Sequence: Synthetic polynucleotide”
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, Artificial Sequence
  source: /note=“Description of Artificial Sequence: Synthetic polynucleotide”
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, Artificial Sequence; identical to SEQ ID NO: 29
  source: /note=“Description of Artificial Sequence: Synthetic polypeptide”

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, Artificial Sequence
  source: /note=“Description of Artificial Sequence: Synthetic polynucleotide”
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, Artificial Sequence; identical to SEQ ID NO: 36
  source: /note=“Description of Artificial Sequence: Synthetic polypeptide”

SEQ ID NO: 39-47 - skipped (9 entries)

SEQ ID NO: 48 - protein, 452 aa, Artificial Sequence
  source: /note=“Description of Artificial Sequence: Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Artificial Sequence
  source: /note=“Description of Artificial Sequence: Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 446 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 120 aa, Artificial Sequence [sequence omitted]

SEQ ID NO: 53 - protein, 111 aa, Artificial Sequence [sequence omitted]

SEQ ID NO: 54 - protein, 5 aa, Artificial Sequence
  source: /note=“Description of Artificial Sequence: Synthetic peptide”
NYYMY

SEQ ID NO: 55 - protein, 17 aa, Artificial Sequence
  source: /note=“Description of Artificial Sequence: Synthetic peptide”
GINPSNGGTNFNEKFKN

SEQ ID NO: 56 - protein, 11 aa, Artificial Sequence
  source: /note=“Description of Artificial Sequence: Synthetic peptide”
RDYRFDMGFDY

SEQ ID NO: 57 - protein, 15 aa, Artificial Sequence
  source: /note=“Description of Artificial Sequence: Synthetic peptide”
RASKGVSTSGYSYLH

SEQ ID NO: 58 - protein, 7 aa, Artificial Sequence
  source: /note=“Description of Artificial Sequence: Synthetic peptide”
LASYLES

SEQ ID NO: 59 - protein, 9 aa, Artificial Sequence
  source: /note=“Description of Artificial Sequence: Synthetic peptide”
QHSRDLPLT

SEQ ID NO: 60-69 - skipped (10 entries)

SEQ ID NO: 70 - protein, 131 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARDSRGYSYYDFWSGYFYYYYMDVWGKGTTVTVSS

SEQ ID NO: 71 - protein, 113 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPWASYTFGQGTKLEIKRT

SEQ ID NO: 72 - protein, 141 aa, Artificial Sequence
  source: /note=“Description of Artificial Sequence: Synthetic polypeptide”
MAWVWTLLFLMAAAQSIQAQVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 73 - protein, 127 aa, Artificial Sequence
  source: /note=“Description of Artificial Sequence: Synthetic polypeptide”
MESQIQVFVFVFLWLSGVDGDIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 74 - protein, 136 aa, Homo sapiens
GLSWVFLVAILEGVQCEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGLTGATDAFDIWGQGTMVTVSS

SEQ ID NO: 75 - protein, 128 aa, Homo sapiens
MEAPAQLLFLLLLWLPDTTGEIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 76 - protein, 139 aa, Artificial Sequence
  source: /note=“Description of Artificial Sequence: Synthetic polypeptide”
MDFGLSLVFLVLILKSVQCEVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 77 - protein, 131 aa, Artificial Sequence
  source: /note=“Description of Artificial Sequence: Synthetic polypeptide”
METDTLLLWVLLLWVPGSTGEIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 78-97 - skipped (20 entries)

SEQ ID NO: 98 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 113 aa, Artificial Sequence [sequence omitted]

SEQ ID NO: 101 - protein, 107 aa, Artificial Sequence [sequence omitted]

SEQ ID NO: 102 - protein, 5 aa, Artificial Sequence
  source: /note=“Description of Artificial Sequence: Synthetic peptide”
NSGMH

SEQ ID NO: 103 - protein, 17 aa, Artificial Sequence
  source: /note=“Description of Artificial Sequence: Synthetic peptide”
VIWYDGSKRYYADSVKG

SEQ ID NO: 104 - protein, 4 aa, Artificial Sequence
  source: /note=“Description of Artificial Sequence: Synthetic peptide”
NDDY

SEQ ID NO: 105 - protein, 11 aa, Artificial Sequence
  source: /note=“Description of Artificial Sequence: Synthetic peptide”
RASQSVSSYLA

SEQ ID NO: 106 - protein, 7 aa, Artificial Sequence
  source: /note=“Description of Artificial Sequence: Synthetic peptide”
DASNRAT

SEQ ID NO: 107 - protein, 9 aa, Artificial Sequence
  source: /note=“Description of Artificial Sequence: Synthetic peptide”
QQSSNWPRT